(19) United States
(12) Patent Application Publication
(10) Pub. No.: US 2004/0081652 A1
(43) Pub. Date: Apr. 29, 2004

(54) Neuronal and optic nerve gene expression patterns

(75) Inventors: Donald Jeffery Zack, Baltimore, MD (US); Harry A. Quigley, Baltimore, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD

(21) Appl. No.: 10/617,888

(22) Filed: Jul. 14, 2003

Related U.S. Application Data
(60) Provisional application No. 60/395,821, filed on Jul. 15, 2002.

(51) Int. Cl. A61K 39/395

(52) U.S. Cl. 424/146.1; 424/145.1

(57) ABSTRACT

The optic nerve axotomy model in mouse exhibits rapid changes in gene expression. Genes identified by microarray analysis as differentially expressed or modulated in this model, can be used diagnostically, therapeutically, and in drug discovery. These results provide clues to underlying molecular processes occurring during optic nerve degeneration, and provide direction for future cell-based studies.

This application claims the benefit of provisional application serial No. 60/395,821 filed Jul. 15, 2002, the disclosure of which is expressly incorporated herein.

### TECHNICAL FIELD OF THE INVENTION

This invention is related to the area of neuronal cell death. In particular, it relates to genes which are characteristically dyregulated in neuronal cells, including the optic nerve, when subjected to a lethal challenge.

### BACKGROUND OF THE INVENTION

The molecular events contributing to optic nerve disease are highly complex. Naturally occurring and experimentally generated rodent models have been valuable for exploring the functional and histopathological changes that occur in many diseases, including optic nerve diseases. One well-studied model is optic nerve axotomy. This experimental manipulation causes retinal ganglion cell death. Retinal ganglion cell death is responsible for visual loss in a number of optic nerve disease, including glaucoma. Optic nerve axotomy is an extremely useful model, therefore, for glaucoma.

There is a continuing need in the art to characterize degenerating or dying neuronal cells relative to normal neuronal cells so that any differences can be exploited for therapeutic and diagnostic benefits.

### SUMMARY OF THE INVENTION

A first embodiment of the invention provides a method for inhibiting neuronal cell death in a mammalian subject. An effective amount of an isolated molecule comprising an antibody variable region is administered to a subject in need thereof. The antibody variable region specifically binds to a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. Neuronal cell death is thereby inhibited.

A second embodiment of the invention provides a method for preventing neuronal cell death in a mammal. A nucleic acid molecule comprising a coding sequence for a NM protein is administed to the mammal. The coding sequence is selected from the group consisting of: NM androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+ von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-1); ACVRLK2, calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1 B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2×5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta I subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytyptamine (serotonin) receptor 1B; 5HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP). Neuronal cell death in the mammal is thereby inhibited or prevented.

A third embodiment of the invention is a method for preventing neuronal cell death in a mammal. A purified human NM protein selected from the group consisting of: NM androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CD C25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha IC subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node horning receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP 17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATPIB1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2×5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1, (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein I precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP) is administered to the mammal. Neuronal cell death in the mammal is thereby inhibited or prevented.

A fourth embodiment of the invention is a method of identifying regions of neuronal cell death in a patient. A molecule comprising an antibody variable region is administered to the patient. The molecule is bound to a detectable moiety. The antibody variable region specifically binds to a NM protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1);

gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. The detectable moiety in the patent is detected. Regions of neuronal cell death are thereby detected.

A fifth embodiment of the invention is a method of screening for neuronal cell death in a patient. A body fluid collected from the patient is contacted with a molecule comprising an antibody variable region which specifically binds to a NM protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2. Detection of cross-reactive material in the body fluid with the molecule indicates neuronal cell death in the patient.

A sixth embodiment of the invention is method for promoting neuronal cell death in a patient. An NM protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A is administered to the patient. Neuronal cell death in the patient is thereby stimulated.

A seventh embodiment of the invention is a method of promoting neuronal cell death in a patient. A nucleic acid molecule encoding a NM protein is administered to the patient. The NM protein is selected from the group consising of microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. The NM protein is expressed in the patient and neuronal cell death in the patient is thereby stimulated.

An eighth embodiment of the invention is a method of screening for neuronal cell death in a patient. An NM protein is detected in a body fluid collected from the patient. The NM protein is selected from the group consisting of microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. Detection of the NM protein indicates neuronal cell death in the patient.

A ninth embodiment of the invention is a method of screening for neuronal cell death in a patient. A nucleic acid encoding an NM protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A is detected in a body fluid of the patient. Detection of the NM protein indicates neuronal cell death in the patient.

A tenth embodiment of the invention is a method to identify candidate drugs for treating neuronal cell death. Cells which express one or more NM genes are contacted with a test compound. The NM genes are selected from the group consisting of microglobulin; beta2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. Expression of said one or more NM genes is detected by hybridization of mRNA of said cells to a nucleic acid probe which is complementary to said mRNA. A test compound is identified as a candidate drug for treating neuronal cell death if it decreases expression of said one or more NM genes.

An eleventh embodiment of the invention is a method to identify candidate drugs for treating neuronal cell death. Cells which express one or more NM proteins are contacted with a test compound. The NM proteins are selected from the group consisting of:

microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. The amount of said one or more NM proteins in said cells is determined. A test compound is identified as a candidate drug for treating tumors if it decreases the amount of one or more NM proteins in said cells.

An eleventh embodiment of the invention is a method to identify candidate drugs for treating neuronal cell death. Cells which express one or more NM proteins are contacted with a test compound. The NM proteins are selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A. Activity of said one or more NM proteins in said cells is determined. A test compound is identified as a candidate drug for treating neuronal cell death if it decreases the activity of one more NM proteins in said cells.

A twelfth embodiment of the invention is a method to identify candidate drugs for treating neuronal cell death. Cells are contacted with a test compound. The cells express one or more NM genes selected from the group consisting of androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATPIB1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2×5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B, gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP). Expression of said one or more N\M genes is detected by hybridization of mRNA of said cells to a nucleic acid probe which is complementary to said mRNA. A test compound is identified as a candidate drug for treating neuronal cell death if it increases expression of said one or more NM genes.

A thirteenth embodiment of the invention is a method for identifying candidate drugs for treating neuronal cell death. Cells which express one or more NM proteins are contacted with a test compound. The NM proteins are selected from the group consisting of: androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha IC subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4 A1; AE1; L-selectin precursor; lymph node homing receptor; leukocyte adhesion molecule-1 (LAM1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MvP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2×5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein VISININ-LIKE PROTEIN 1 (VILIP-1) (NEURAL VISININ-LIKE PROTEIN 1) (NVL-1) (NVP-1) (21 KD CABP). The amount of said one or more NM proteins in said cells is determined. A test compound is identified as a candidate drug for treating neuronal cell death if it increases the amount of one more NM proteins in said cells.

A fourteenth embodiment of the invention is a method to identify candidate drugs for treating neuronal cell death. Cells are contacted with a test compound. The cells express one or more NM proteins selected from the group consisting of: androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 A1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha IC subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2X3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytyptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2X5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL1) (NVP-1) (21 KD CABP). Activity of said one or more NM proteins in said cells is determined. A test compound is identified as a candidate drug for treating neuronal cell death if it increases the activity of one more NM proteins in said cells.

Any of the methods of the present invention can also be performed using any of the genes indentified as AF, AS. BF, and BS, as shown in FIG. 8. These and other embodiments which will be apparent to those of skill in the art upon reading the specification provide the art with reagents and methods for detection, diagnosis, therapy, and drug screening pertaining to neuronal cell death and pathological processes involving or requiring neuronal cell death.

### BRIEF DESCRIPTION OF THE DRAWINGS

FIG. 1 shows genes which were down-regulated at day 1 after axiotomy, comparing one eye to the other in each animal.

FIG. 2 shows genes which were up-regulated at day 3 after axiotomy, comparing one eye to the other in each animal.

FIG. 3 shows genes which were down-regulated at day 3 after axiotomy, comparing one eye to the other in each animal.

FIG. 4 shows genes which were up-regulated at day 7 after axiotomy, comparing one eye to the other in each animal.

FIG. 5 shows genes which were down-regulated at day 7 after axiotomy, comparing one eye to the other in each animal.

FIG. 6 shows genes which were up-regulated at day 14 after axiotomy, comparing one eye to the other in each animal.

FIG. 7 shows genes which were down-regulated at day 14 after axiotomy, comparing one eye to the other in each animal.

FIG. 8 shows genes whose expression was modulated using tests AF, AS, BF, andn BS. These tests compared treated rats with a single axiotomy to control rats with no axiotomy.

FIG. 9 shows the names of genes whose numbers are referenced in FIG. 9.

### DETAILED DESCRIPTION OF THE INVENTION

The inventors have identified gene expression changes that may mediate neuronal disease progression, in particular, the cell death of retinal ganglion cells. Neurodegenerative disorders can be treated and identified using the methods of the present invention. These include disorders of the central nervous system as well as disorders of the peripheral nervous system. Neurodegenerative disorders include, but are not limited to, brain injuries, cerebrovascular diseases and their consequences, Parkinson's disease, corticobasal degeneration, motor neuron disease (including ALS), multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias (including Pick's disease), progressive nuclear palsy, corticobasal degeneration, Huntington's disease, thalamic degeneration, Creutzfeld-Jakob dementia, HIV dementia, schizophrenia with dementia, and Korsakoffs psychosis, also are neurodegenerative disorders.

Neuronal cell death is a major feature of a variety of human neurological disorders, including the neurodegenerative diseases (such as Alzheimer's, Parkinson's, Huntington's and amyotrophic lateral sclerosis), stroke and trauma. Alzheimer's Disease afflicts about 4 million people in the United States, primarily the elderly. It is characterized by progressive memory loss, disorientation, depression and eventual loss of bodily functions. Amyotrophic lateral sclerosis, afflicts about 30,000 Americans. It begins after age 40 and results in progressive weakness and paralysis. Huntington's Disease, which afflicts an estimated 25,000 patients in the United States, usually begins between the ages of 30 and 50 and includes violent, involuntary movements.

Loss of neurons by a degenerative process is a major pathological feature of many human neurological disorders. Neuronal cell death can occur as a result of a variety of conditions including traumatic injury, ischemia, neurodegenerative diseases (e.g., Parkinson's disease, Huntington's disease, Alzheimer's disease, amyotrophic lateral sclerosis (ALS), stroke, or trauma), or as a normal part of tissue development and maintenance. Several inherited disorders produce late onset neuron loss, each of which is highly specific for particular neural cell types.

The methods of the present invention can be applied to any of the diseases of the optic nerve, the retina, retinal pigment epithelium (RPE), and choroid. These include, but are not limited to, glaucoma, ocular neovascularization, ocular inflammation and retinal degenerations. Specific examples of these disease states include diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, retinitis pigmentosa, retinal vein occlusion, proliferative vitreoretinopathy, angioid streak, and retinal artery occlusion, and, neovascularization due to penetration of the eye or ocular injury. Additional relevant disease include the neuropathies, such as Leber's, idiopathic, drug-induced, optic, and ischemic neropathies.

Any type of neuronal cells can be used in the practice of the invention, for example, for screening for candicate drugs for treating neuronal cell death and disease resulting therefrom. Such cells include without limitation cells isolated from brain, neuroblastoma, astrocytoma, glioblastoma, medulloblastoma, retinoblastoma, and retina. Such cells can be isolated as is known in the art. Cell lines of these types are available from the American Type Culture Collection, Mannassas, Va. Cells that can differentiate into neurons, such as NT2, and PC12 cells can also be used to advantage.

Isolated and purified nucleic acids, according to the present invention are those which are not linked to those genes to which they are linked in the human genome. Moreover, isolated and purified nucleic acids are not present in a mixture, such as a library, containing a multitude of distinct sequences from distinct genes. They may be, however, linked to other genes such as vector sequences or sequences of other genes to which they are not naturally adjacent. The nucleic acids may represent either the sense or the antisense strand. Nucleic acids and proteins although disclosed herein with sequence particularity may be derived from a single individual. Allelic variants which occur in the population of humans are including within the scope of such nucleic acids and proteins. Those of skill in the art are well able to identify allelic variants as being the same gene or protein.

Isolated and purified proteins are not in a cell, and are separated from the normal cellular constituents, such as nucleic acids, lipids, etc. Typically the protein is purified to such an extent that it comprises the predominant species of protein in the composition, such as greater than 50, 60 70, 80, 90, or even 95% of the proteins present.

Using the proteins according to the invention, one of ordinary skill in the art can readily generate or obtain antibodies which specifically bind to the proteins. Such antibodies can be monoclonal or polyclonal. They can be chimeric, humanized, or totally human. Any functional fragment or derivative of an antibody can be used including Fab, Fab′, Fab2, Fab′2, and single chain variable regions. So long as the fragment or derivative retains specificity of binding for the endothelial marker protein it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one such technique, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Nina D. Russel, Jose R. F. Corvalan, Michael L. Gallo, C. Geoffrey Davis, Liise-Anne Pirofski. Production of Protective Human Antipneumococcal Antibodies by Transgenic Mice with Human Immunoglobulin Loci Infection and Immunity April 2000, p. 1820-1826; Michael L. Gallo, Vladimir E. Ivanov, Aya Jakobovits, and C. Geoffrey Davis. The human immunoglobulin loci introduced into mice: V (D) and J gene segment usage similar to that of adult humans European Journal of Immunology 30: 534-540, 2000; Larry L. Green. Antibody engineering via genetic engineering of the mouse: XenoMouse strains are a vehicle for the facile generation of therapeutic human monoclonal antibodies Journal of Immunological Methods 231 11-23, 1999; Yang X-D, Corvalan JRF, Wang P, Roy CM-N and Davis C G. Fully Human Anti-interleukin-8 Monoclonal Antibodies: Potential Therapeutics for the Treatment of Inflammatory Disease States. Journal of Leukocyte Biology Vol. 66, pp401-410 (1999); Yang X-D, Jia X-C, Corvalan J R F, Wang P, C G Davis and Jakobovits A. Eradication of Established Tumors by a Fully Human Monoclonal Antibody to the Epidermal Growth Factor Receptor without Concomitant Chemotherapy. Cancer Research Vol. 59, Number 6, pp1236-1243 (1999); Jakobovits A. Production and selection of antigen-specific fully human monoclonal antibodies from mice engineered with human Ig loci. Advanced Drug Delivery Reviews Vol. 31, pp: 33-42 (1998); Green L and Jakobovits A. Regulation of B cell development by variable gene complexity in mice reconstituted with human immunoglobulin yeast artificial chromosomes. J. Exp. Med. Vol. 188, Number 3, pp: 483-495 (1998); Jakobovits A. The long-awaited magic bullets: therapeutic human monoclonal antibodies from transgenic mice. Exp. Opin. Invest. Drugs Vol. 7(4), pp: 607-614 (1998); Tsuda H, Maynard-Currie K, Reid L, Yoshida T, Edamura K, Maeda N, Smithies O, Jakobovits A. Inactivation of Mouse HPRT locus by a 203-bp retrotransposon insertion and a 55-kb gene-targeted deletion: establishment of new HPRT-Deficient mouse embryonic sNM cell lines. Genomics Vol. 42, pp: 413-421 (1997); Sherman-Gold, R. Monoclonal Antibodies: The Evolution from '80s Magic Bullets To Mature, Mainstream Applications as Clinical Therapeutics. Genetic Engineering News Vol. 17, Number 14 (August 1997); Mendez M, Green L, Corvalan J, Jia X-C, Maynard-Currie C, Yang X-d, Gallo M, Louie D, Lee D, Erickson K, Luna J, Roy C, Abderrahim H, Kirschenbaum F, Noguchi M, Smith D, Fukushima A, Hales J, Finer M, Davis C, Zsebo K, Jakobovits A. Functional transplant of megabase human immunoglobulin loci recapitulates human antibody response in mice. Nature Genetics Vol. 15, pp: 146-156 (1997); Jakobovits A. Mice engineered with human immunoglobulin YACs: A new technology for production of fully human antibodies for autoimmunity therapy. Weir's Handbook of Experimental Immunology, The Integrated Immune SysNM Vol. IV, pp: 194.1-194.7 (1996); Jakobovits A. Production of fully human antibodies by transgenic mice. Current Opinion in Biotechnology Vol. 6, No. 5, pp: 561-566 (1995); Mendez M, Abderrahim H, Noguchi M, David N, Hardy M, Green L, Tsuda H, Yoast S, Maynard-Currie C, Garza D, Gemmill R, Jakobovits A, Klapholz S. Analysis of the structural integrity of YACs comprising human immunoglobulin genes in yeast and in embryonic sNM cells. Genomics Vol. 26, pp: 294-307 (1995); Jakobovits A. YAC Vectors: Humanizing the mouse genome. Current Biology Vol. 4, No. 8, pp: 761-763 (1994); Arbones M, Ord D, Ley K, Ratech H, Maynard-Curry K, Otten G, Capon D, Tedder T. Lymphocyte homing and leukocyte rolling and migration are impaired in L-selectin-deficient mice. Immunity Vol. 1, No. 4, pp: 247-260 (1994); Green L, Hardy M, Maynard-Curry K, Tsuda H, Louie D, Mendez M, Abderrahim H, Noguchi M, Smith D, Zeng Y, et. al. Antigen-specific human monoclonal antibodies from mice engineered with human Ig heavy and light chain YACs. Nature Genetics Vol. 7, No. 1, pp: 13-21 (1994); Jakobovits A, Moore A, Green L, Vergara G, Maynard-Curry K, Austin H, Klapholz S. Germ-line transmission and expression of a human-derived yeast artificial chromosome. Nature Vol. 362, No. 6417, pp: 255-258 (1993); Jakobovits A, Vergara G, Kennedy J, Hales J, McGuinness R, Casentini-Borocz D, Brenner D, Otten G. Analysis of homozygous mutant chimeric mice: deletion of the immunoglobulin heavy-chain joining region blocks B-cell development and antibody production. Proceedings of the National Academy of Sciences USA Vol. 90, No. 6, pp: 2551-2555 (1993); Kucherlapati et al., U.S. Pat. No. 6,1075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fingi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti-tumor agents (e.g., antisense oligonucleotides, plasmids which encode for toxins, methotrexate, etc.); and other antibodies or antibody fragments, such as F(ab).

Those of skill in the art will readily understand and be able to make such antibody derivatives, as they are well known in the art. The antibodies may be cytotoxic on their own, or they may be used to deliver cytotoxic agents to particular locations in the body. The antibodies can be administered to individuals in need thereof as a form of passive immunization.

Drugs can be screened for the ability to modulate expression of the genes, mRNA, and protein which are identified herein. Cell populations can be contacted with test substances and the expression of neuronal cell death markers determined. Test substances which decrease the expression of up-regulated neuronal cell death markers are candidates for inhibiting neuronal cell death. Conversely, test substances which increase the expression of down-regulated neuronal cell death markers can be identified as candidate drugs for causing neuronal cell death. In cases where a biological or enzymatic activity of a NM is known, agents can be screened for their ability to decrease or increase the activity or amount of activity present in a cell.

Expression can be monitored according to any convenient method. Protein or mRNA can be monitored. Any technique known in the art for monitoring specific genes' expression can be used, including but not limited to ELISAs, SAGE, custom or commercial microarray hybridization, Western blots. Changes in expression of a single marker may be used as a criterion for significant effect as a potential pro-neuronal cell death or anticell death agent. However, it also may be desirable to screen for test substances which are able to modulate the expression of groups of such markers, such as modulators of at least 5, 10, 15, or 20 of the relevant markers. Inhibition of NM protein activity can also be used as a drug screen.

Neuronal cell death markers identified herein were identified using available reagents for probes. In some cases these probes are human. In other case they derive from other mammalian species. Each gene has an ortholog in humans, and the human ortholog is to be used for treating humans. When cells, cell lines, and whole animal models of other species are used, it is preferred that the corresponding ortholog be used. Nonetheless, as demonstrated in the examples below, probes for orthologs of other species can be used.

Test substances for screening can come from any source. They can be from libraries of natural products, combinatorial chemical libraries, biological products made by recombinant libraries, etc. The source of the test substances is not critical to the invention. The present invention provides means for screening compounds and compositions which may previously have been overlooked in other screening schemes.

Nucleic acids and the corresponding encoded proteins of the markers of the present invention can be used therapeutically in a variety of modes. The nucleic acids and encoded proteins can be administered by any means known in the art. Such methods include, using liposomes, nanospheres, viral vectors, non-viral vectors comprising polycations, etc. Suitable viral vectors include adenovirus, retroviruses, and sindbis virus. Administration modes can be any known in the art, including parenteral, intravenous, intramuscular, intraperitoneal, topical, intranasal, intrarectal, intrabronchial, etc. Such administrations can be used to reduce or eliminate cell death (down-regulated genes or proteins) or induce cell death (up-regulated genes or proteins). The pathological condition of the patient will determine which type of gene or protein should be used.

Specific biological antagonists of NMs can also be used to therapeutic benefit. For example, antibodies, T cells specific for an NM, antisense to an NM, and ribozymes specific for an NM can be used to restrict, inhibit, reduce, and/or diminish neuronal cell death (up-regulated genes or proteins). Conversely, antagonists of down-regulated genes or proteins can be used to induce or stimulated neuronal cell death. Such antagonists can be administered as is known in the art for these classes of antagonists generally.

Mouse counterparts to human NMs can be used in mouse models or in cell lines or in vitro to evaluate potential anti-neuronal cell death or pro-neuronal cell death compounds or therapies. Their expression can be monitored as an indication of effect.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

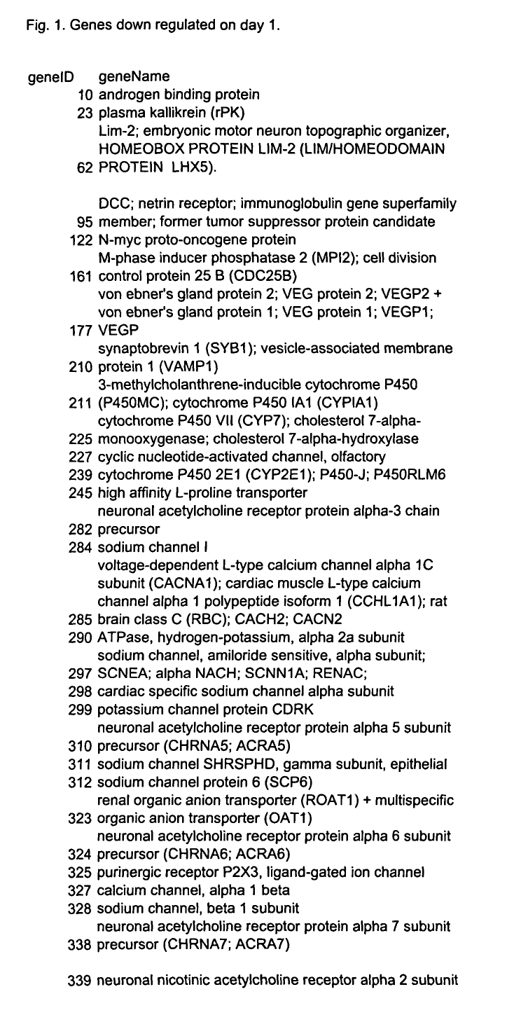

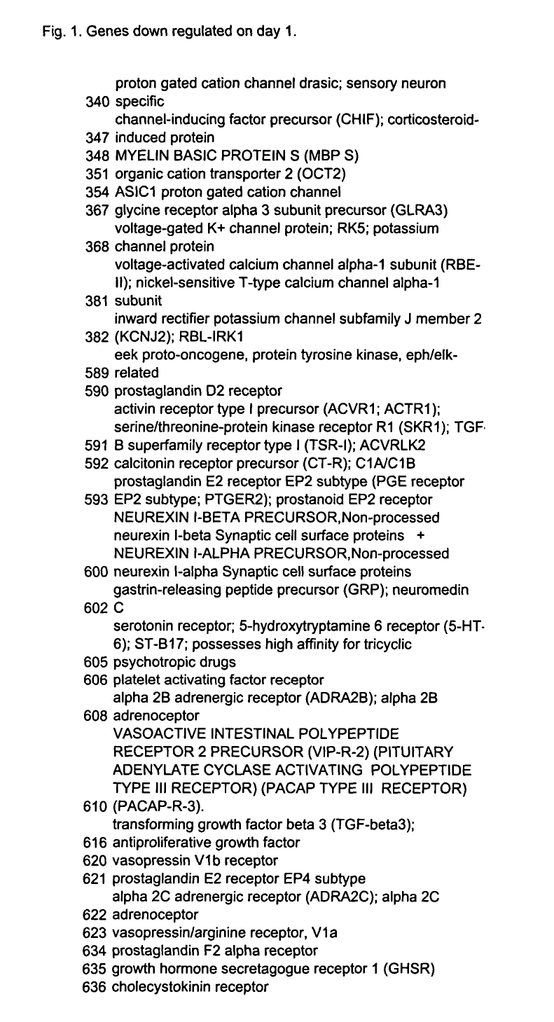

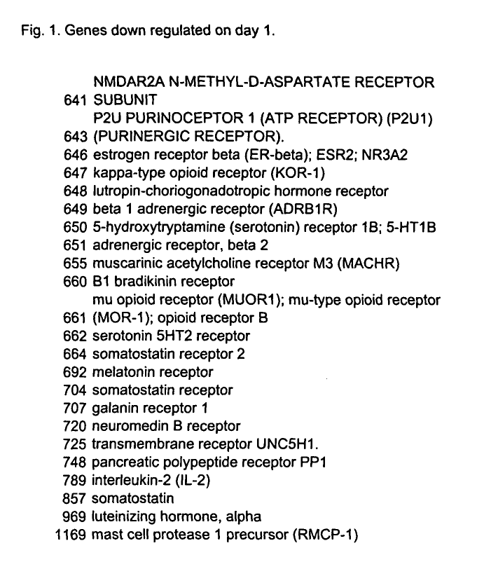

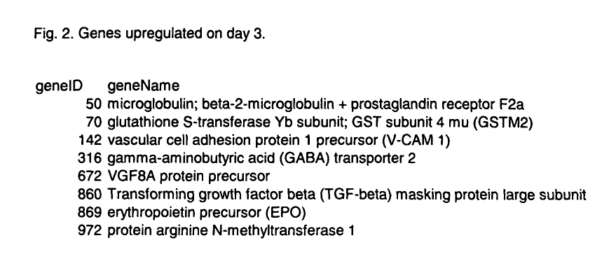

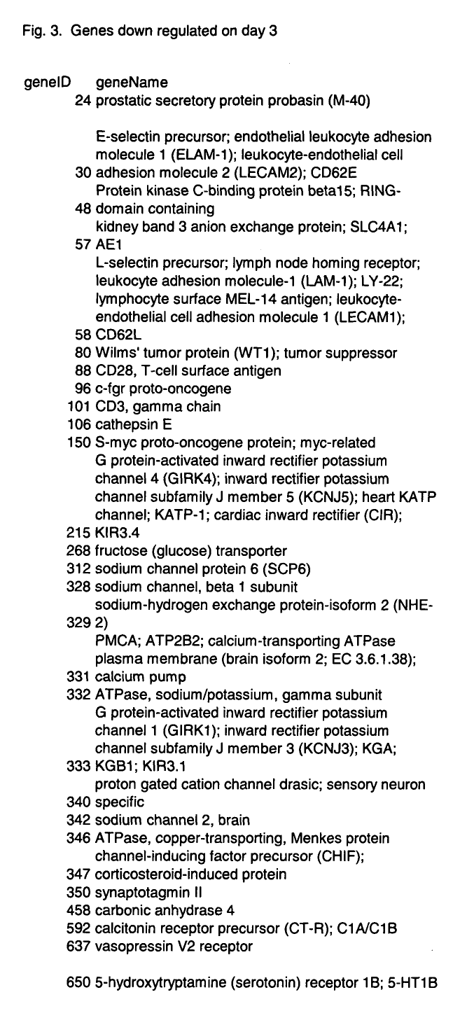

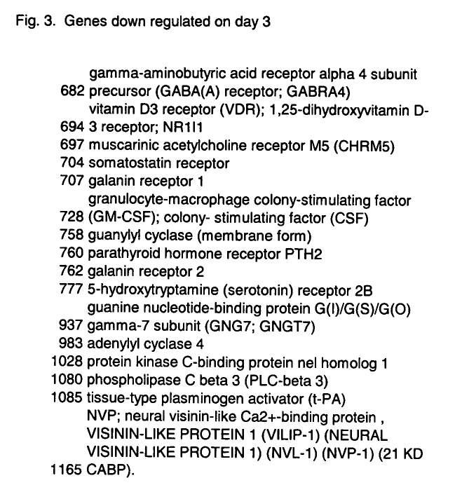

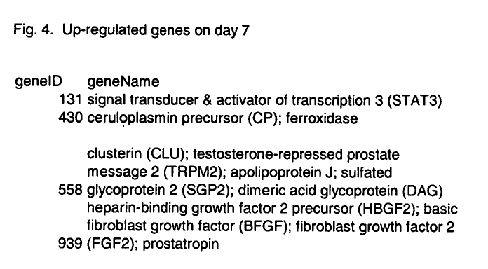

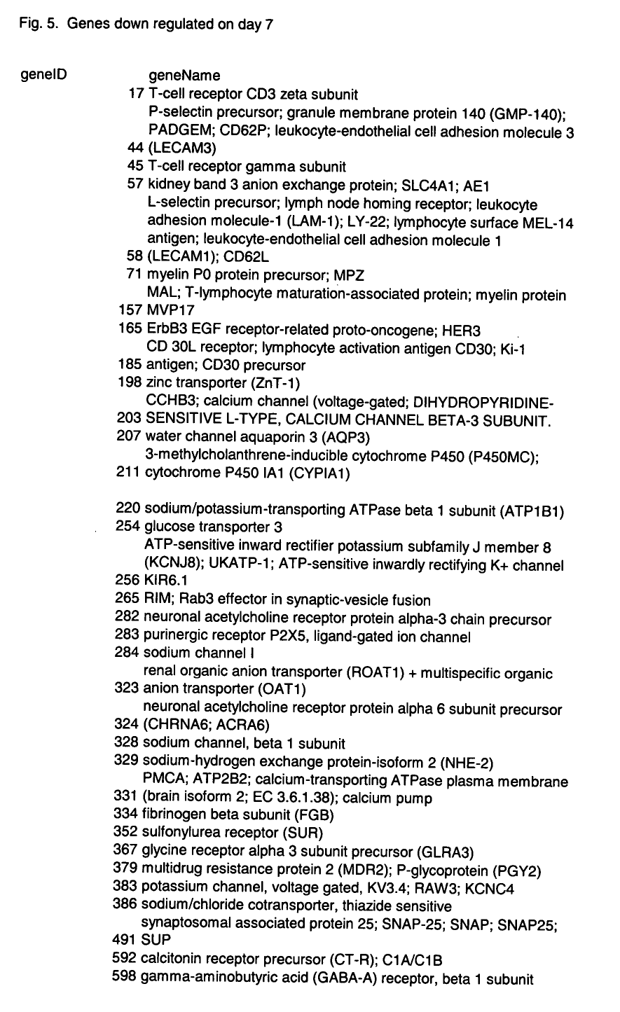

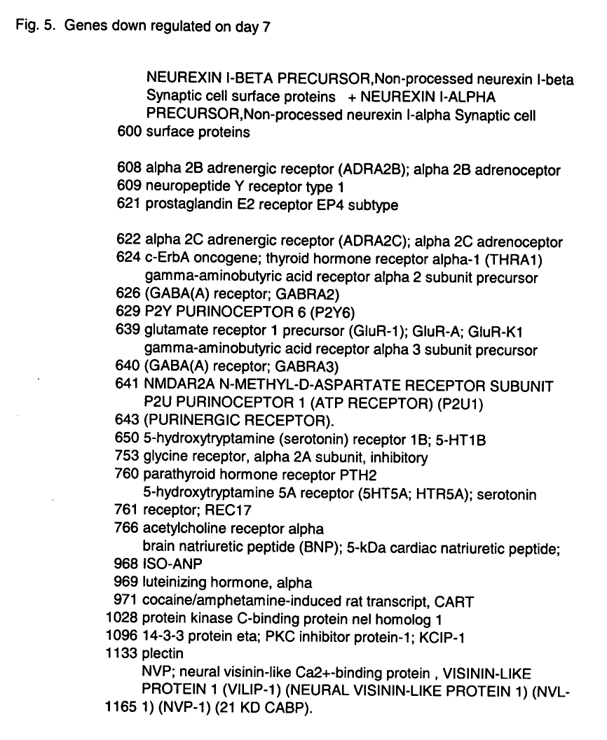

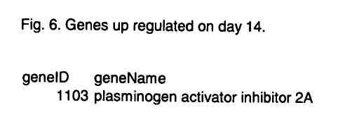

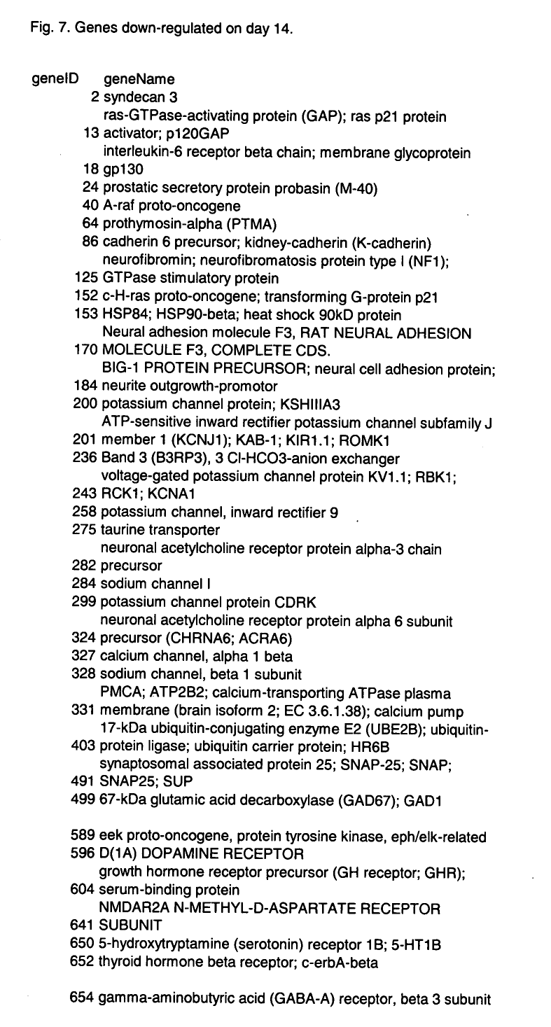

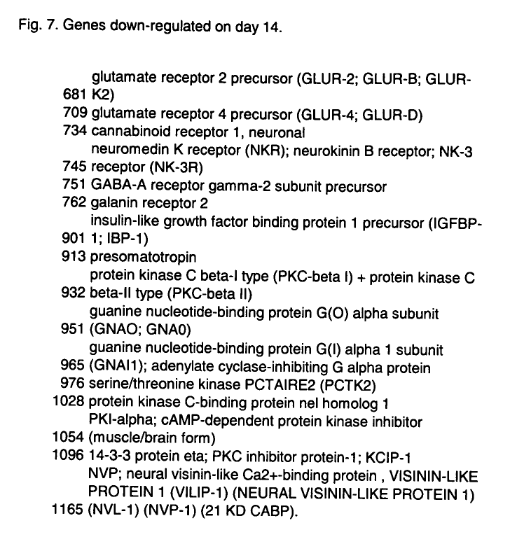

One optic nerve on each rat was severed. Gene expression was assessed using arrays of probes for genes as shown in FIG. 9. By comparing right to left eye, genes were identified that were either up or down regulated at various times after axotomy. See FIGS. 1-7.

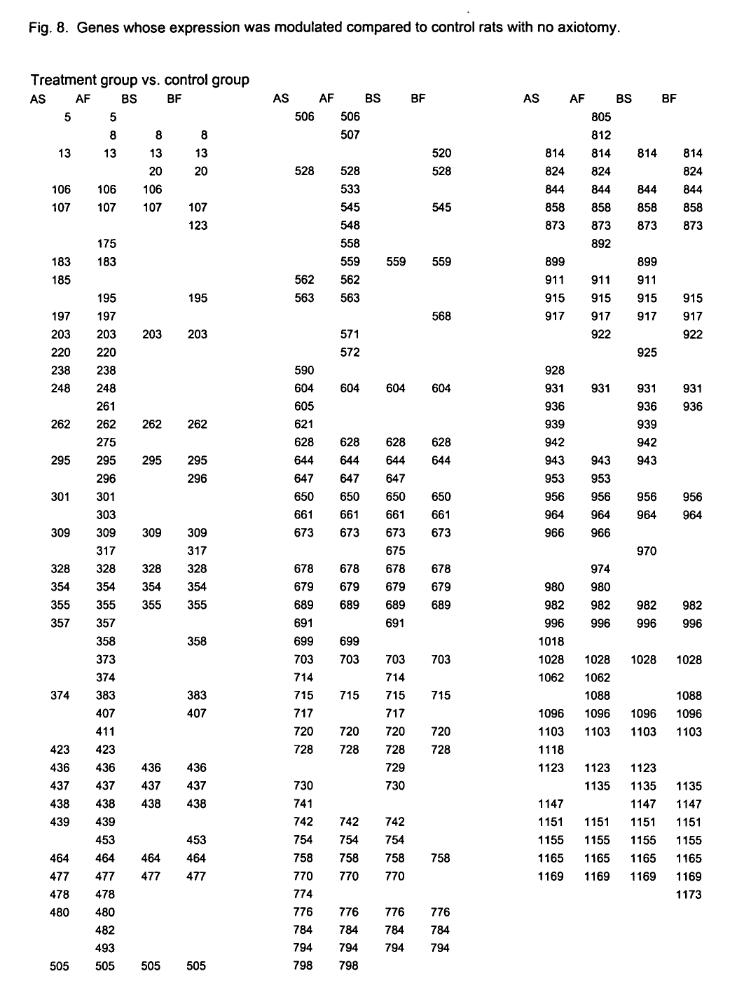

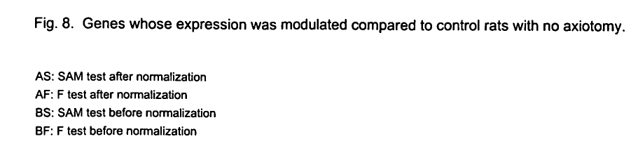

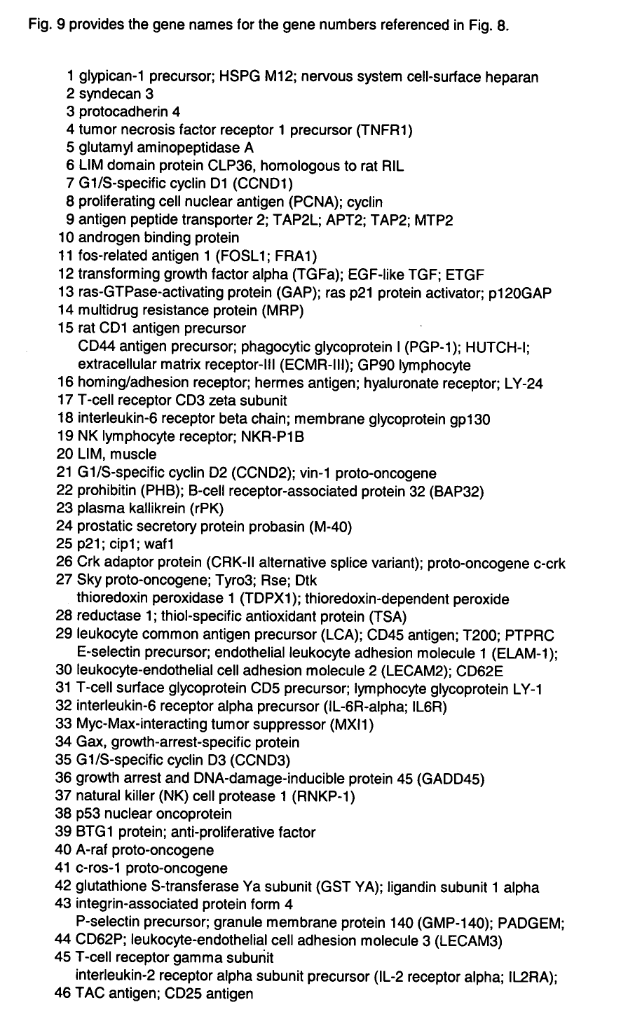

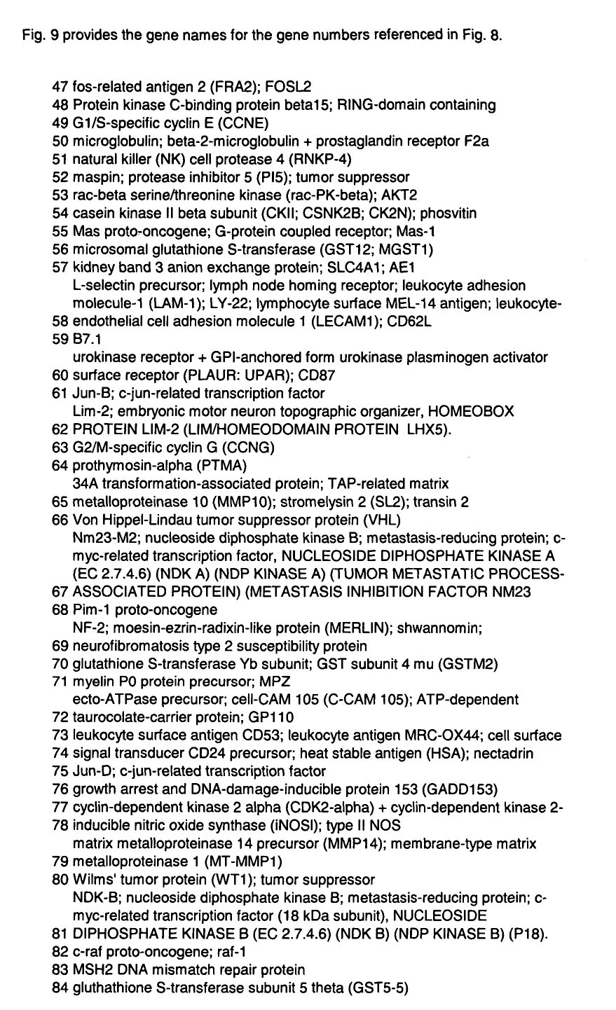

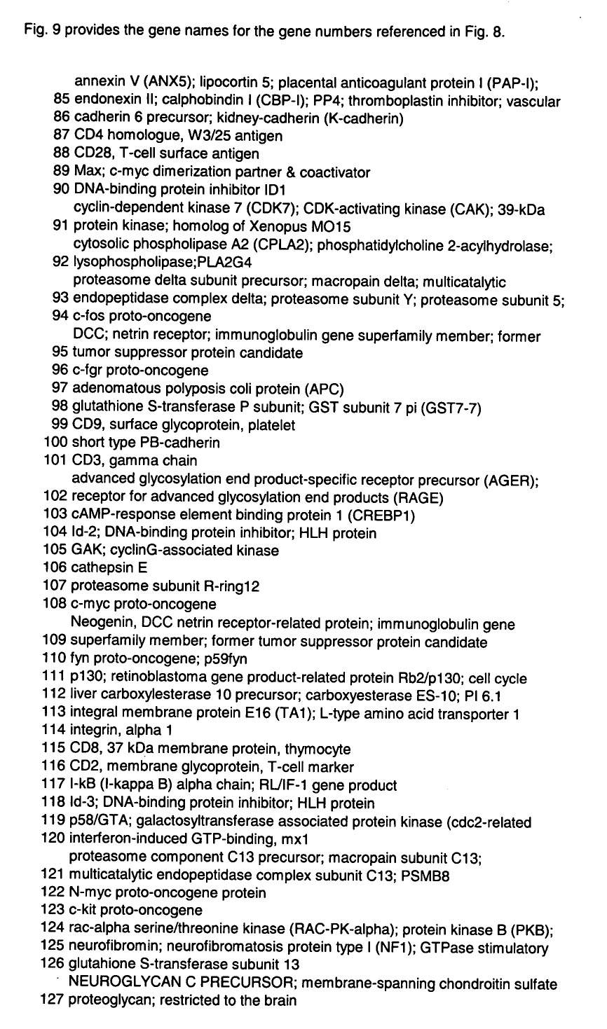

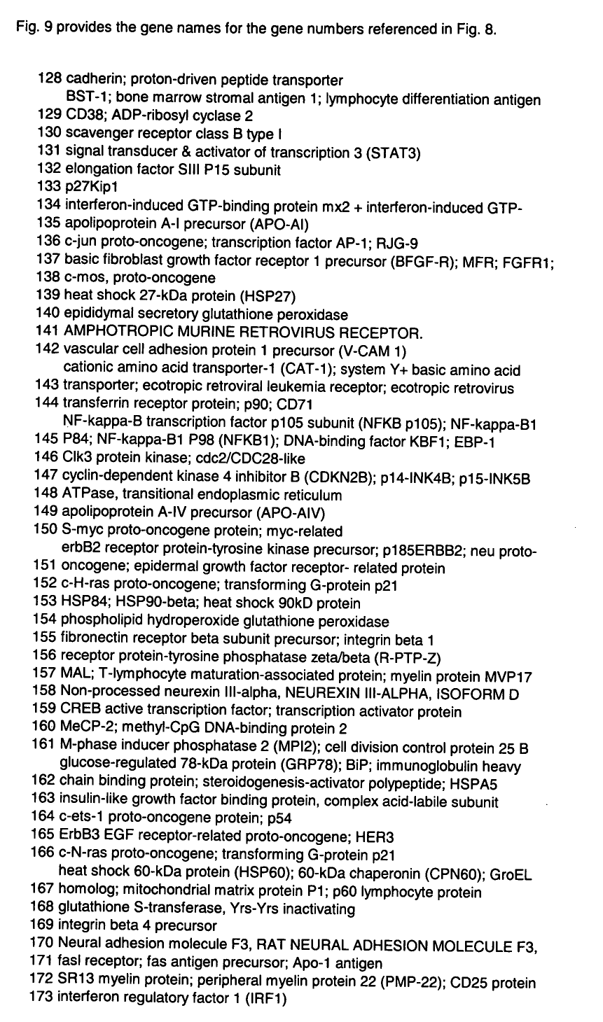

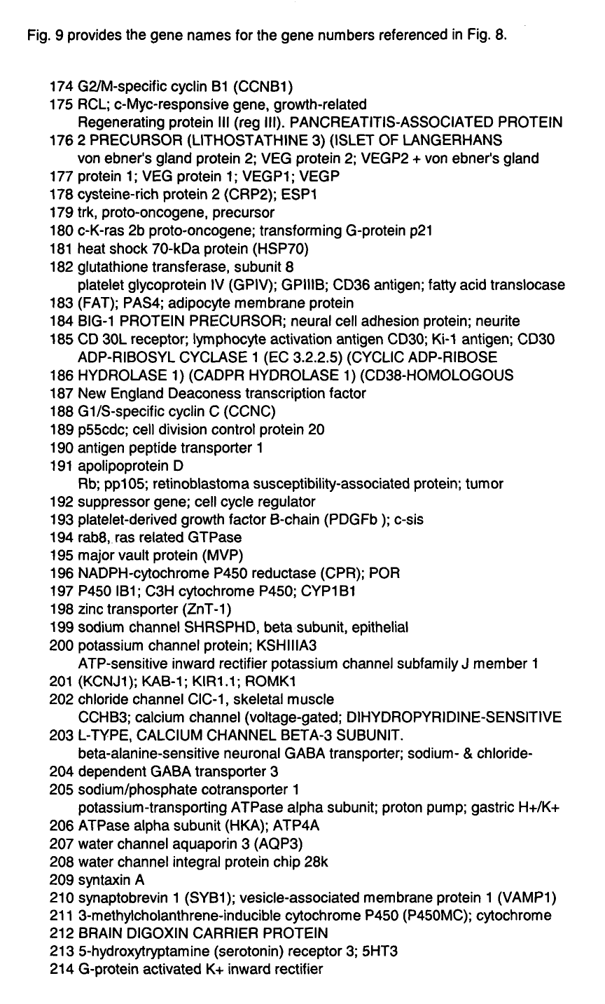

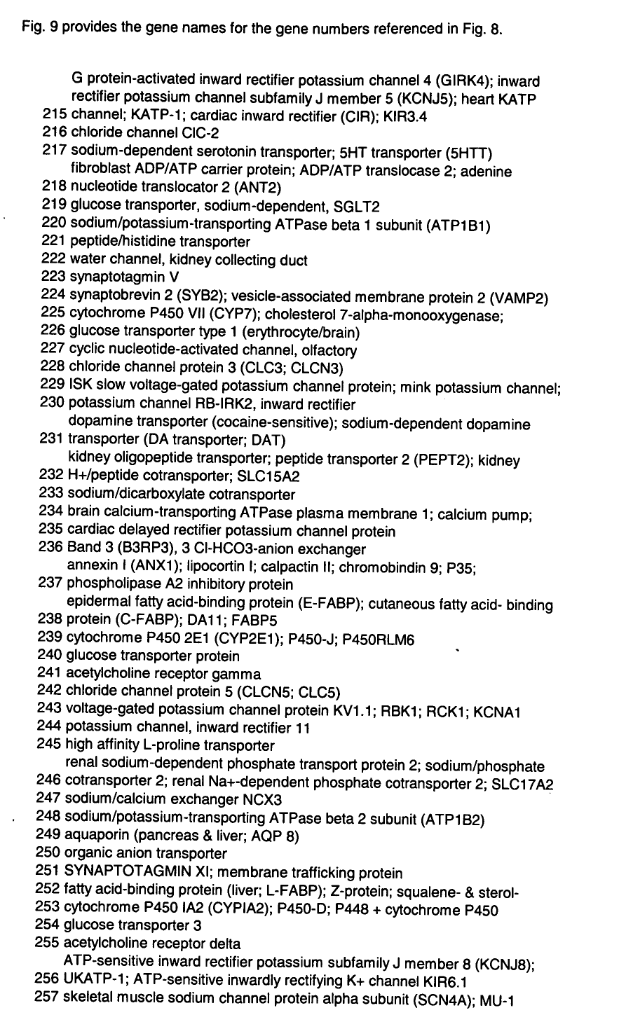

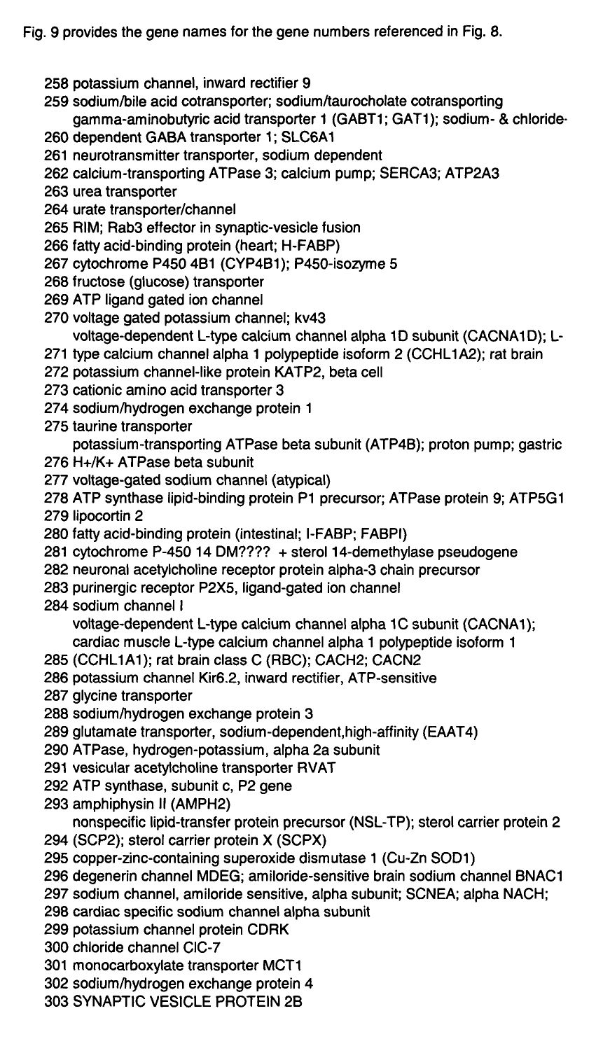

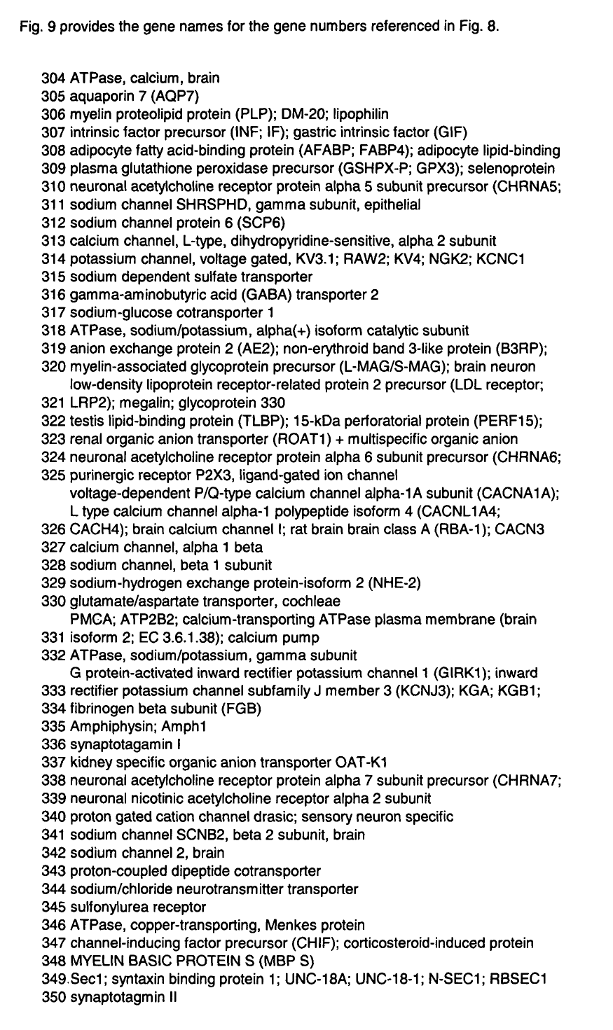

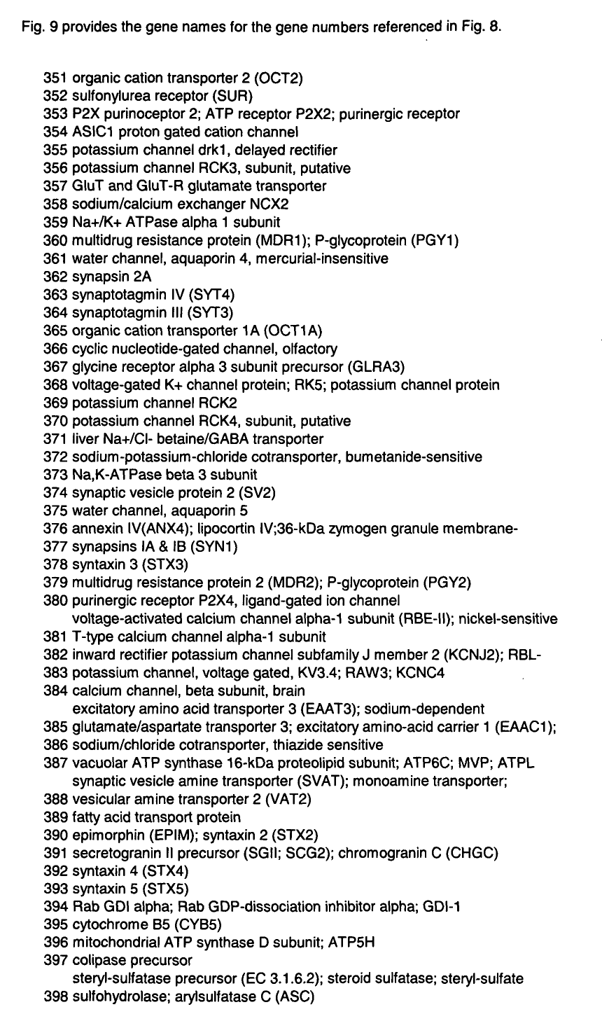

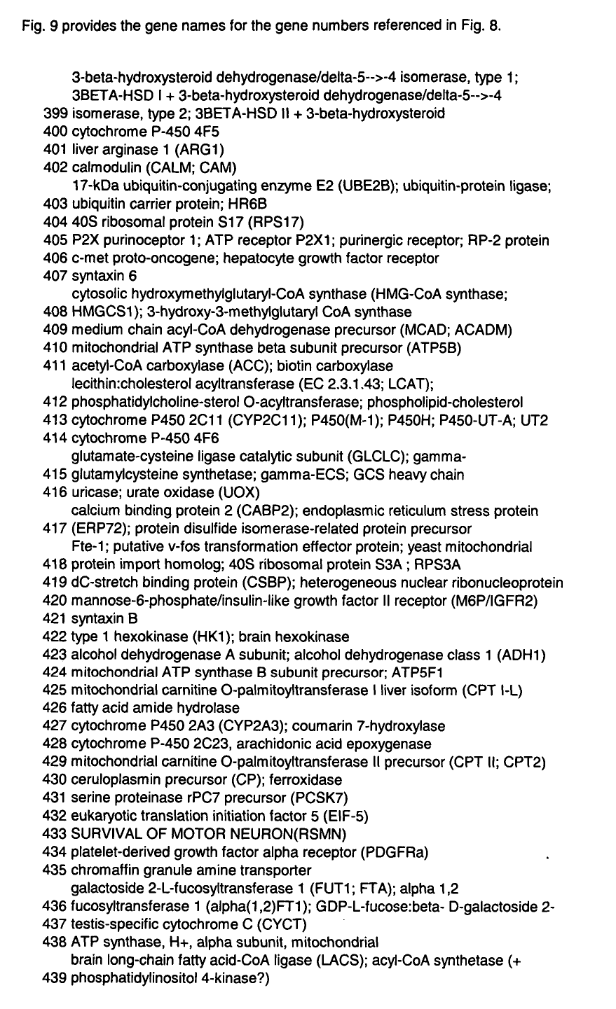

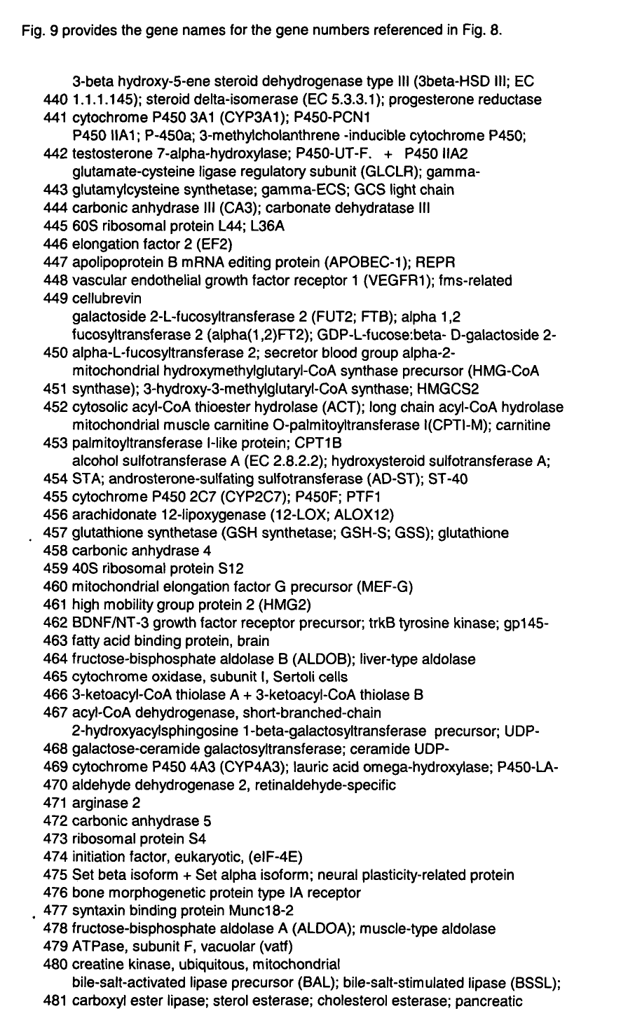

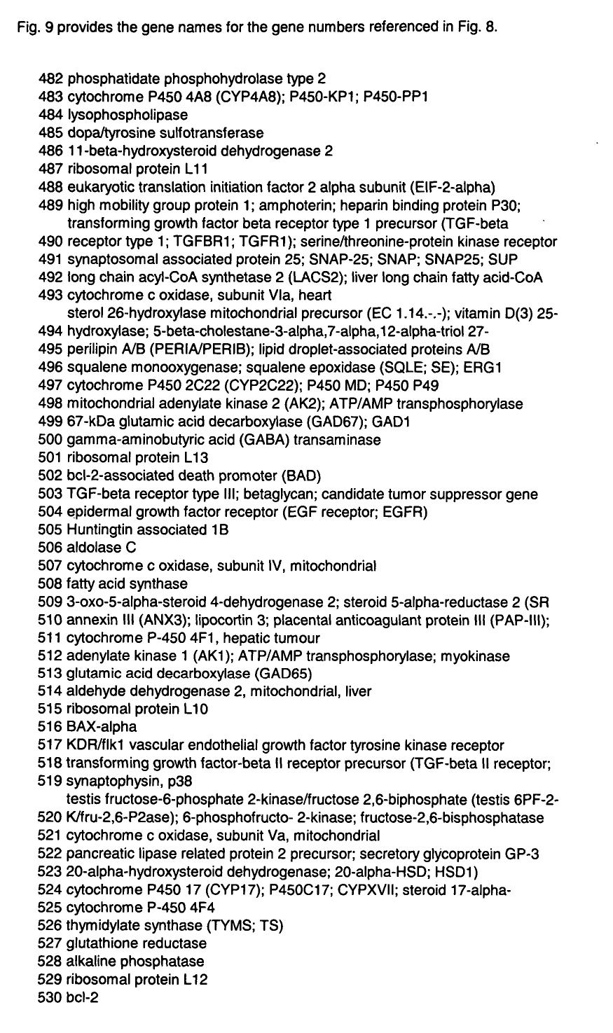

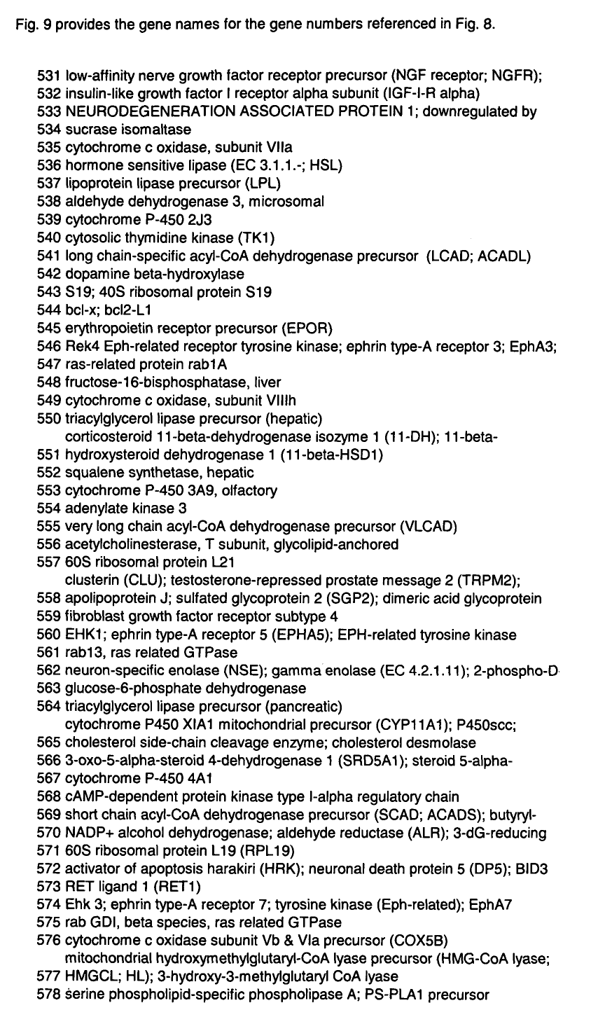

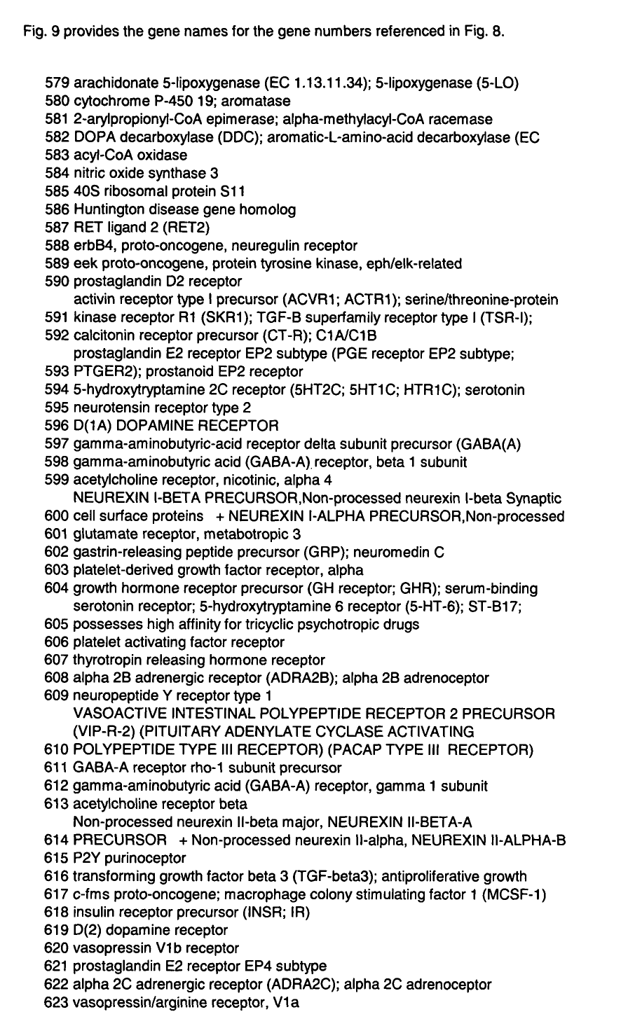

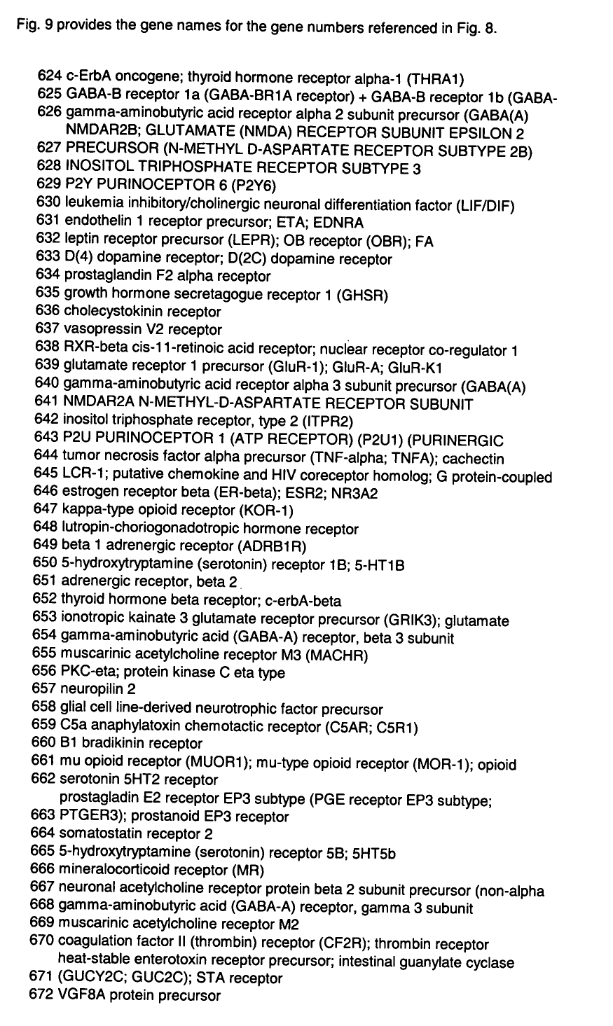

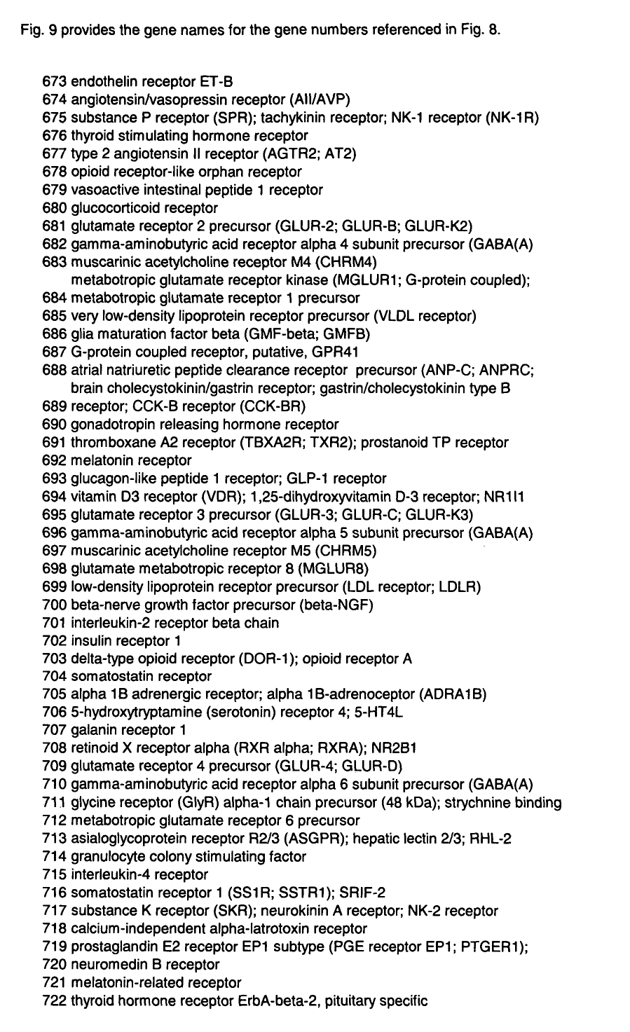

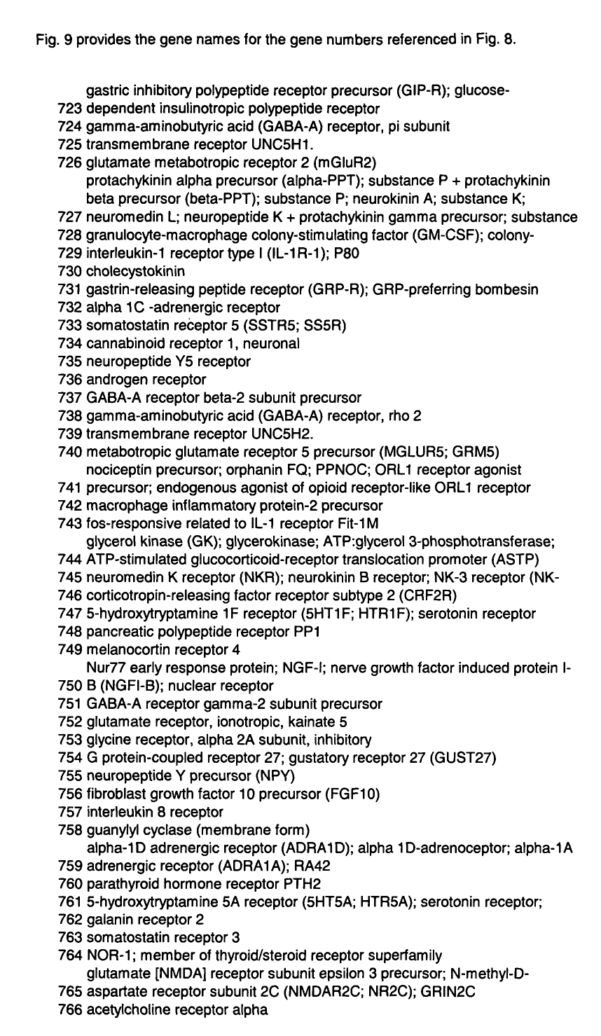

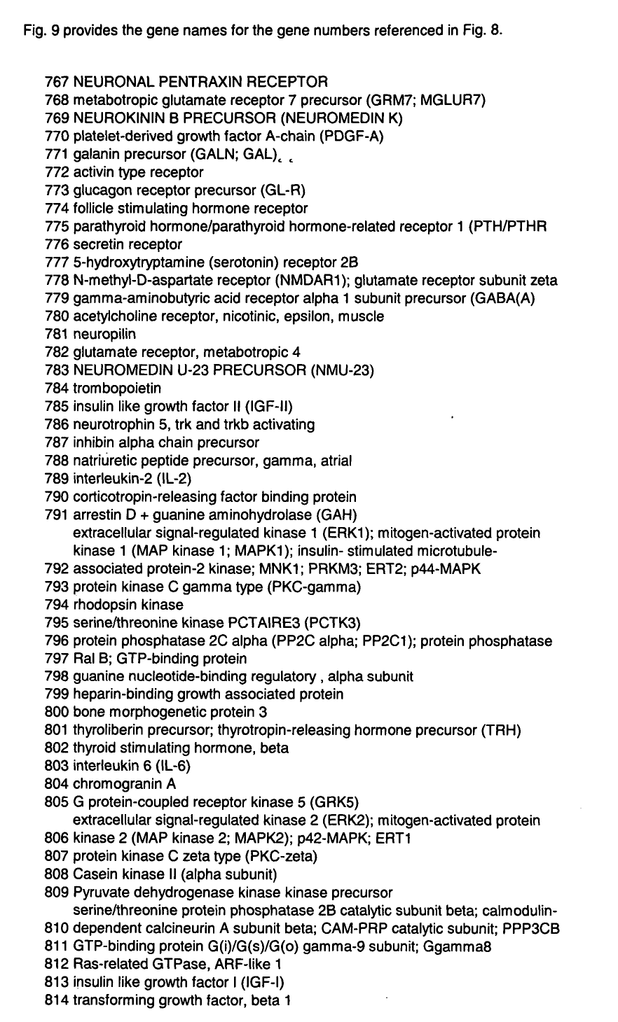

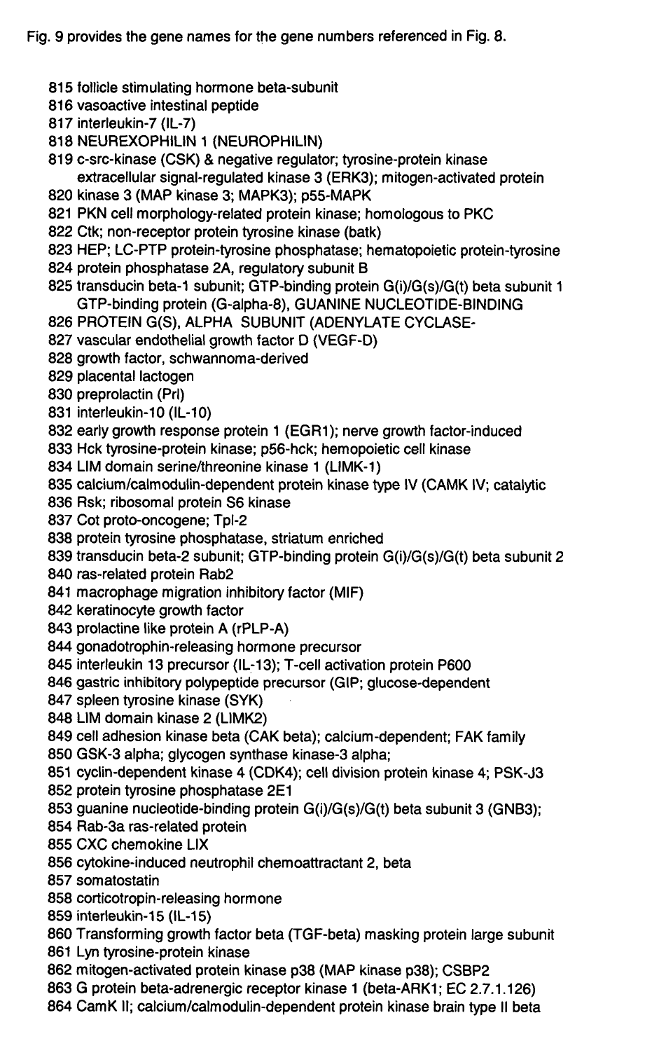

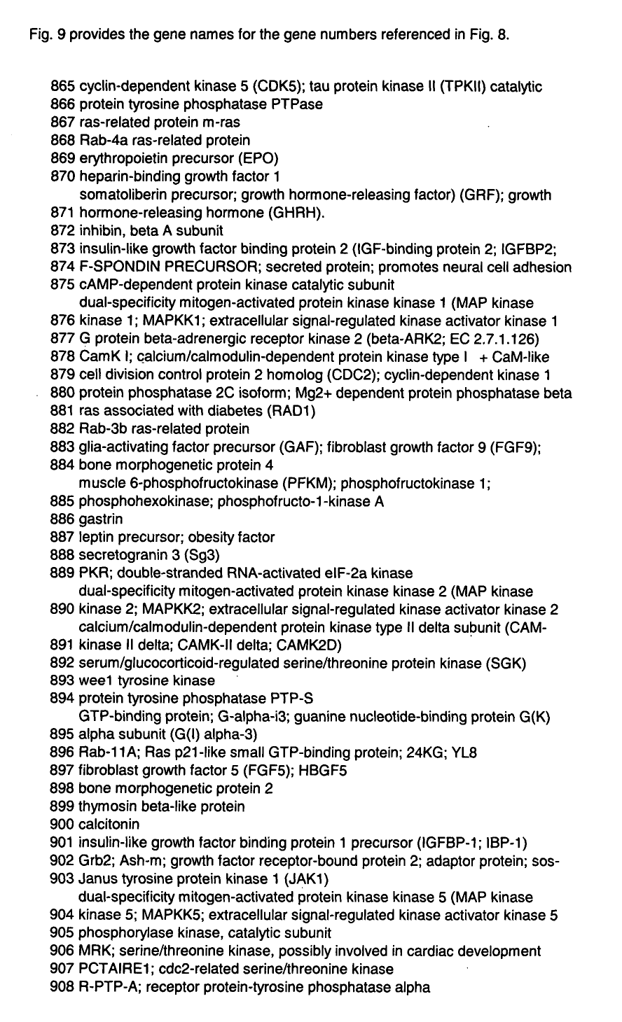

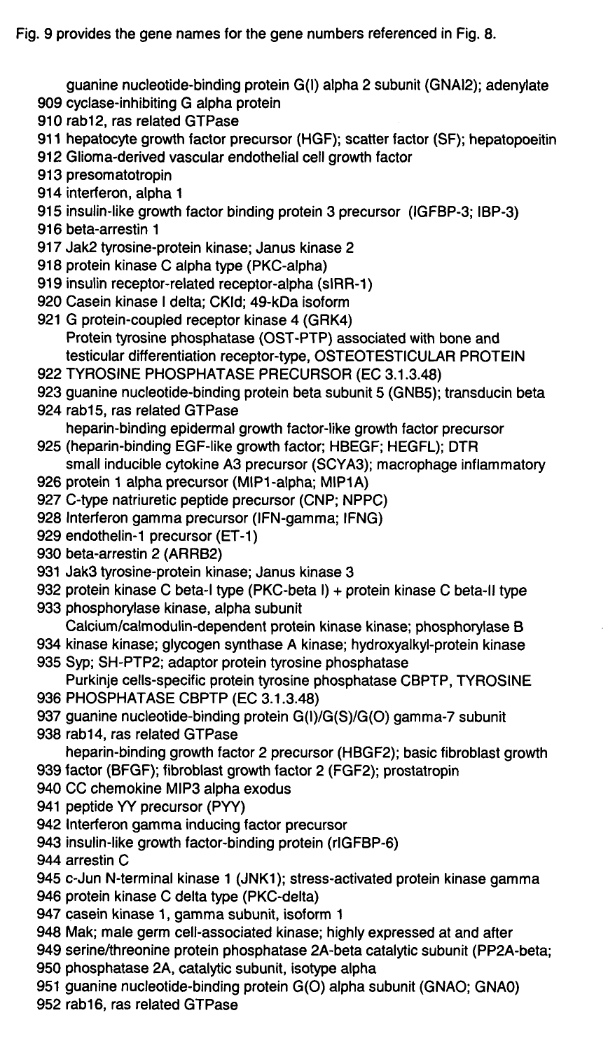

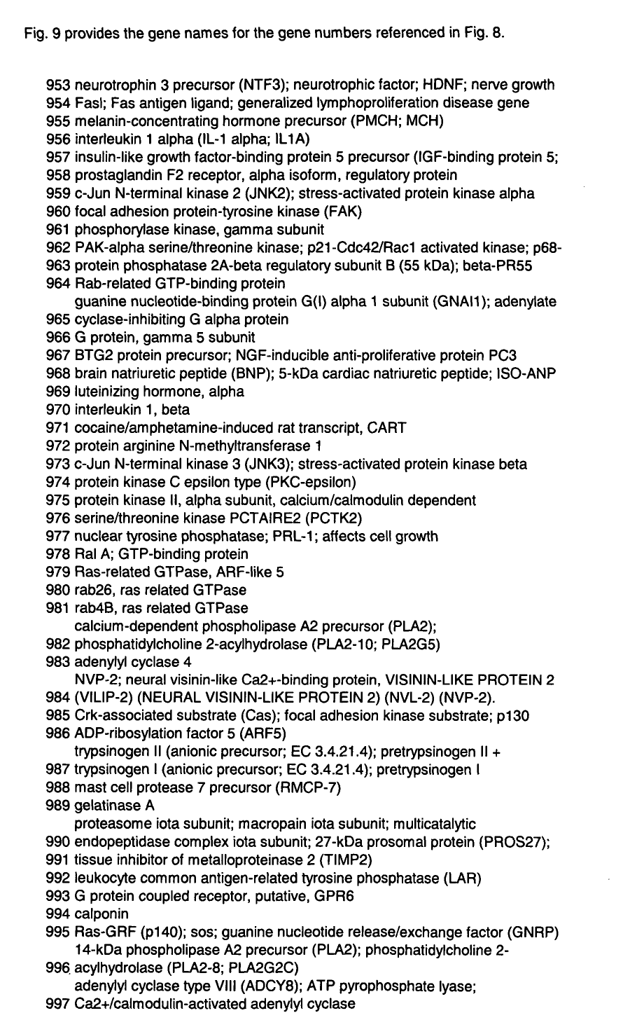

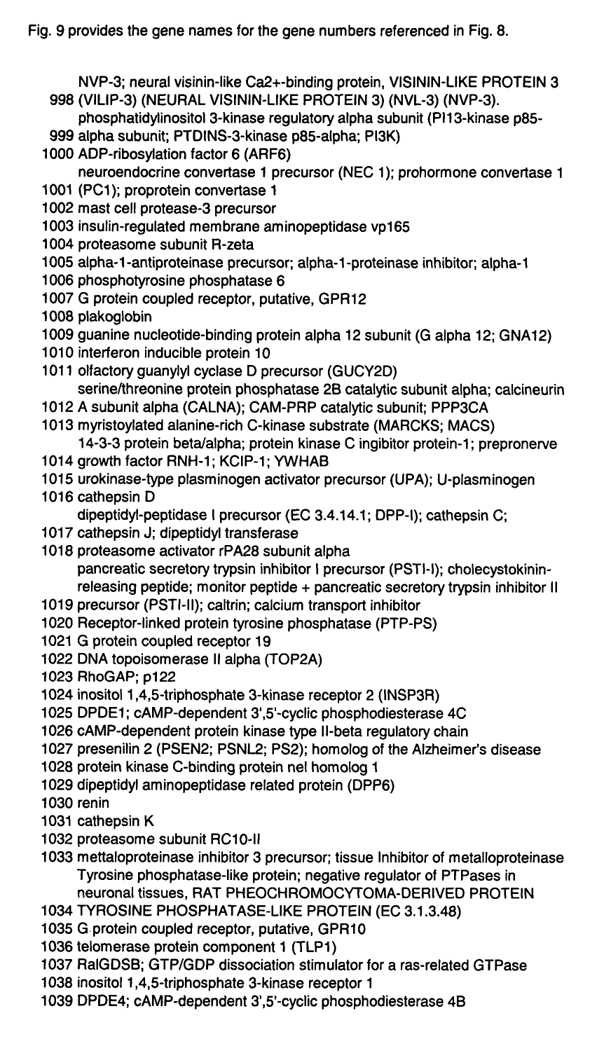

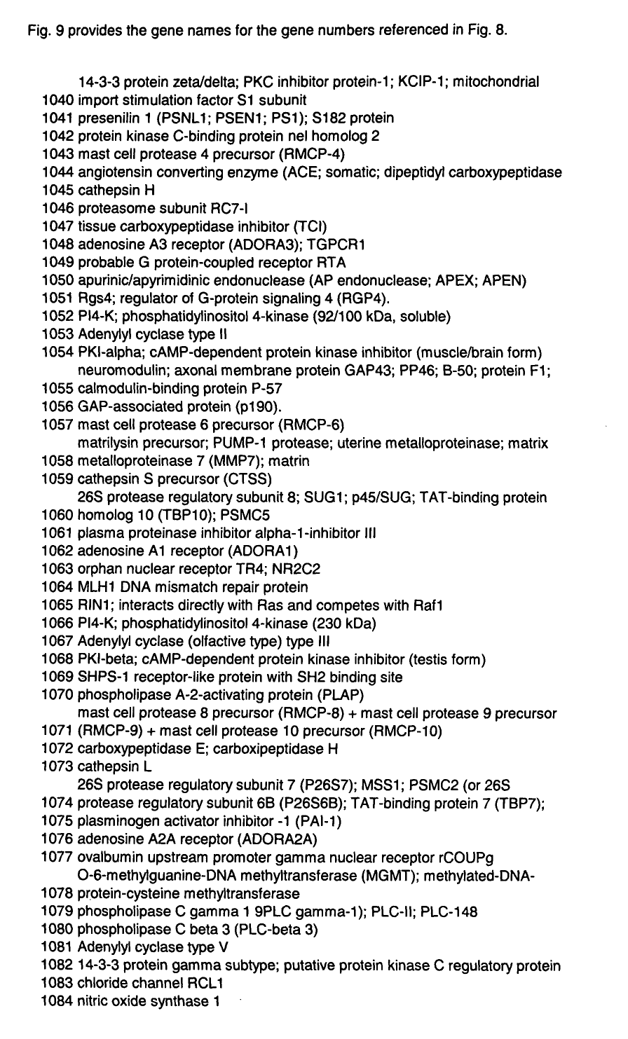

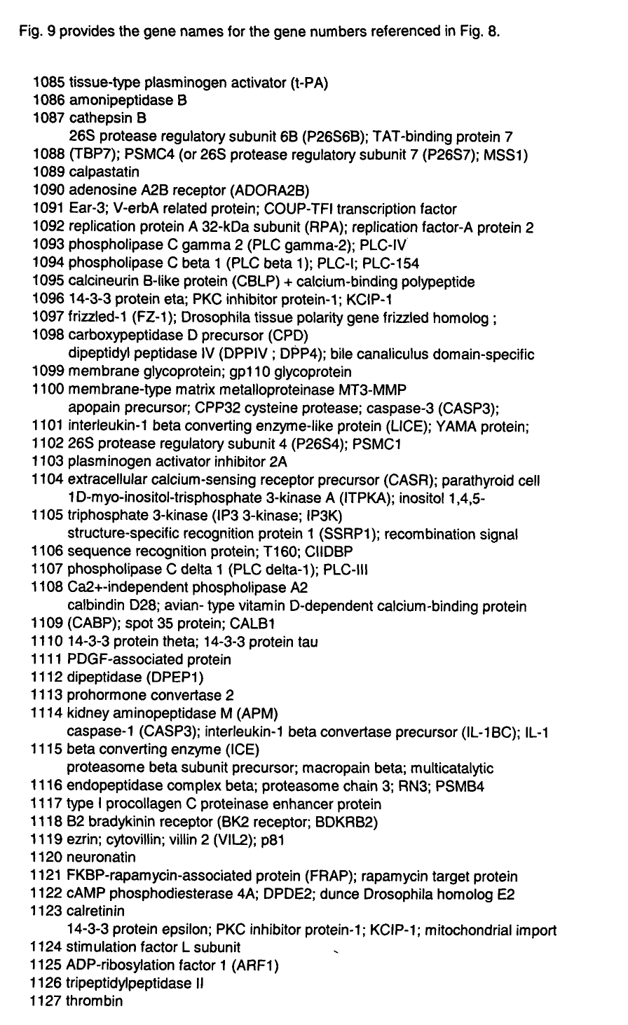

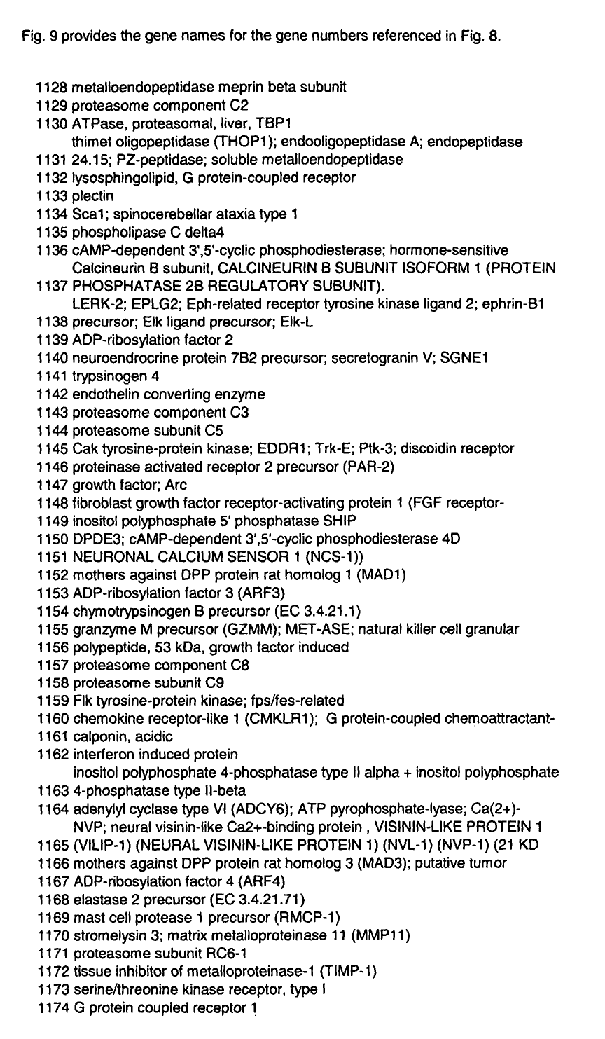

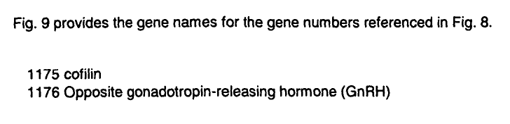

By comparing expression in treated and non-treated rats genes were identified whose expression was modulated relative to the control animals. These are identified in FIG. 8. These genes can be used similarly to those identified in FIGS. 1-7 in any of the methods of the present invention.

1. A method of inhibiting neuronal cell death, comprising:
administering to a subject in need thereof an effective amount of an isolated molecule comprising an antibody variable region which specifically binds to a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, whereby neuronal cell death is inhibited.

2. The method of claim 1 wherein the subject has optic nerve degeneration.

3. The method of claim 1 wherein the subject has Alzheimer's disease.

4. The method of claim 1 wherein the subject has diabetic retinopathy.

5. The method of claim 1 wherein the subject has Huntington's disease.

6. The method of claim 1 wherein the subject has spinal cord injury.

7. The method of claim 1 wherein the subject has Parkinson's disease.

8. The method of claim 1 wherein the subject has glaucoma.

9. The method of claim 1 wherein the subject has age-related macular degeneration.

10. A method of preventing neuronal cell death in a mammal, comprising:
administering to the mammal a nucleic acid molecule comprising a coding sequence for a neuronal marker (NM) protein selected from the group consisting of: NM androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRBIR); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MvP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2×5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP), whereby neuronal cell death in the mammal is inhibited or prevented.

11. A method of preventing neuronal cell death in a mammal, comprising:
administering to the mammal a purified human neuronal marker (NM) protein selected from the group consisting of: NM androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MP12); cell division control protein 25 B (CDC25B); von ebner's gland protein 2; VEG protein 2; VEGP2+von ebner's gland protein 1; VEG protein 1; VEGP 1; VEGP; synaptobrevin 1 (SYB 1); vesicle-associated membrane protein 1 (VAMP 1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2X3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB 1 R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (yDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2X5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120 GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxyrtryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP), whereby neuronal cell death in the mammal is inhibited or prevented.

12. The method of claim 10 or 11 wherein the subject has optic nerve degeneration.

13. The method of claim 10 or 11 wherein the subject has Alzheimer's disease.

14. The method of claim 10 or 11 wherein the subject has diabetic retinopathy.

15. The method of claim 10 or 11 wherein the subject has Huntington's disease.

16. The method of claim 10 or 11 wherein the subject has spinal cord injury.

17. The method of claim 10 or 11 wherein the subject has Parkinson's disease.

18. The method of claim 10 or 11 wherein the subject has glaucoma.

19. The method of claim 10 or 11 wherein the subject has age-related macular degeneration.

20. A method of identifying regions of neuronal cell death in a patient, comprising:
administering to a patient a molecule comprising an antibody variable region which specifically binds to a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, wherein the molecule is bound to a detectable moiety; and
detecting the detectable moiety in the patient, thereby identifying regions of neuronal cell death.

21. The method of claim 20 wherein the subject has optic nerve degeneration.

22. The method of claim 20 wherein the subject has Alzheimer's disease.

23. The method of claim 20 wherein the subject has diabetic retinopathy.

24. The method of claim 20 wherein the subject has Huntington's disease.

25. The method of claim 20 wherein the subject has spinal cord injury.

26. The method of claim 20 wherein the subject has Parkinson's disease.

27. The method of claim 20 wherein the subject has glaucoma.

28. The method of claim 20 wherein the subject has age-related macular degeneration.

29. A method of screening for neuronal cell death in a patient, comprising:
contacting a body fluid collected from the patient with a molecule comprising an antibody variable region which specifically binds to a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, wherein detection of cross-reactive material in the body fluid with the molecule indicates neuronal cell death in the patient.

30. A method of promoting neuronal cell death in a patient, comprising:
administering to a patient in need of neuronal cell death a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, whereby neuronal cell death in the patient is stimulated.

31. The method of claim 30 wherein the patient has a neuronal tumor.

32. A method of promoting neuronal cell death in a patient, comprising:
administering to a patient in need of neuronal cell death a nucleic acid molecule encoding a neuronal marker (NM) protein selected from the group consisting of microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, whereby the NM protein is expressed and neuronal cell death in the patient is stimulated.

33. The method of claim 32 wherein the patient has a neuronal tumor.

34. A method of screening for neuronal cell death in a patient, comprising:
detecting a neuronal marker (NM) protein selected from the group consisting of microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, in a body fluid collected from the patient, wherein detection of the NM protein indicates neuronal cell death in the patient.

35. A method of screening for neuronal cell death in a patient, comprising:
detecting in a body fluid collected from the patient a nucleic acid encoding a neuronal marker (NM) protein selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, wherein detection of the NM protein indicates neuronal cell death in the patient.

36. A method to identify candidate drugs for treating neuronal cell death, comprising:
contacting cells which express one or more neuronal marker (NM) genes selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, with a test compound;
determining expression of said one or more NM genes by hybridization of mRNA of said cells to a nucleic acid probe which is complementary to said mRNA; and
identifying a test compound as a candidate drug for treating neuronal cell death if it decreases expression of said one or more NM genes.

37. The method of claim 36 wherein the cells are retinal cells.

38. The method of claim 36 wherein the cells are recombinant host cells which are transfected with an expression construct which encodes said one or more NMs.

39. A method to identify candidate drugs for treating neuronal cell death, comprising:
contacting cells which express one or more neuronal marker (NM) proteins selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A, with a test compound;
determining amount of said one or more NM proteins in said cells; and
identifying a test compound as a candidate drug for treating tumors if it decreases the amount of one more NM proteins in said cells.

40. The method of claim 39 wherein the cells are retinal cells.

41. The method of claim 39 wherein the cells are recombinant host cells which are transfected with an expression construct which encodes said one or more NMs.

42. A method to identify candidate drugs for treating neuronal cell death, comprising:
contacting cells which express one or more neuronal marker (NM) proteins selected from the group consisting of: microglobulin; beta-2-microglobulin+prostaglandin receptor F2a; glutathione S-transferase Yb subunit; GST subunit 4 mu (GSTM2); vascular cell adhesion protein 1 precursor (V-CAM 1); gamma-aminobutyric acid (GABA) transporter 2; VGF8A protein precursor; Transforming growth factor beta (TGF-beta) masking protein large subunit; erythropoietin precursor (EPO); protein arginine N-methyltransferase 1; signal transducer & activator of transcription 3 (STAT3); ceruloplasmin precursor (CP); ferroxidase; clusterin (CLU); testosterone-repressed prostate message 2 (TRPM2); apolipoprotein J; sulfated glycoprotein 2 (SGP2); dimeric acid glycoprotein (DAG); heparin-binding growth factor 2 precursor (HBGF2); basic fibroblast growth factor; (BFGF); fibroblast growth factor 2 (FGF2); prostatropin; and plasminogen activator inhibitor 2A with a test compound;
determining activity of said one or more NM proteins in said cells; and
identifying a test compound as a candidate drug for treating neuronal cell death if it decreases the activity of one more NM proteins in said cells.

43. The method of claim 42 wherein the cells are retinal cells.

44. The method of claim 42 wherein the cells are recombinant host cells which are transfected with an expression construct which encodes said one or more NMs.

45. A method to identify candidate drugs for treating neuronal cell death, comprising:
contacting cells which express one or more neuronal marker (NM) genes selected from the group consisting of androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MP12); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP 1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2X3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB 1 R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2X5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP), with a test compound;
determining expression of said one or more N\M genes by hybridization of mRNA of said cells to a nucleic acid probe which is complementary to said mRNA; and
identifying a test compound as a candidate drug for treating neuronal cell death if it increases expression of said one or more NM genes.

46. The method of claim 45 wherein the cells are retinal cells.

47. The method of claim 45 wherein the cells are recombinant host cells which are transfected with an expression construct which encodes said one or more NMs.

48. A method to identify candidate drugs for treating neuronal cell death, comprising:
contacting cells which express one or more neuronal marker (NM) proteins selected from the group consisting of: androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MPI2); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2X3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-1 subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-1); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB1R); 5-hydroxytryptamine (serotonin) receptor 1B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB 1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2X5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV1.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD 1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK-3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP), with a test compound;
determining amount of said one or more NM proteins in said cells; and
identifying a test compound as a candidate drug for treating neuronal cell death if it increases the amount of one more NM proteins in said cells.

49. The method of claim 48 wherein the cells are retinal cells.

50. The method of claim 48 wherein the cells are recombinant host cells which are transfected with an expression construct which encodes said one or more NMs.

51. A method to identify candidate drugs for treating neuronal cell death, comprising:
contacting cells which express one or more neuronal marker (NM) proteins selected from the group consisting of: androgen binding protein; plasma kallikrein (rPK); Lim-2; embryonic motor neuron topographic organizer, HOMEOBOX PROTEIN LIM-2 (LIM/HOMEODOMAIN PROTEIN LHX5).; DCC; netrin receptor; immunoglobulin gene superfamily member; former tumor suppressor protein candidate; N-myc proto-oncogene protein; M-phase inducer phosphatase 2 (MP12); cell division control protein 25 B (CDC25B); von ebner's gland protein 2; VEG protein 2; VEGP2+von ebner's gland protein 1; VEG protein 1; VEGP1; VEGP; synaptobrevin 1 (SYB1); vesicle-associated membrane protein 1 (VAMP1); 3-methylcholanthrene-inducible cytochrome P450 (P450MC); cytochrome P450 IA1 (CYPIA1); cytochrome P450 VII (CYP7); cholesterol 7-alpha-monooxygenase; cholesterol 7-alpha-hydroxylase; cyclic nucleotide-activated channel, olfactory; cytochrome P450 2E1 (CYP2E1); P450-J; P450RLM6; high affinity L-proline transporter; neuronal acetylcholine receptor protein alpha-3 chain precursor; sodium channel I; voltage-dependent L-type calcium channel alpha 1C subunit (CACNA1); cardiac muscle L-type calcium channel alpha 1 polypeptide isoform 1 (CCHL1A1); rat brain class C (RBC); CACH2; CACN2; ATPase, hydrogen-potassium, alpha 2a subunit; sodium channel, amiloride sensitive, alpha subunit; SCNEA; alpha NACH; SCNN1A; RENAC; cardiac specific sodium channel alpha subunit; potassium channel protein CDRK; neuronal acetylcholine receptor protein alpha 5 subunit precursor (CHRNA5; ACRA5); sodium channel SHRSPHD, gamma subunit, epithelial; sodium channel protein 6 (SCP6); renal organic anion transporter (ROAT1)+multispecific organic anion transporter (OAT1); neuronal acetylcholine receptor protein alpha 6 subunit precursor (CHRNA6; ACRA6); purinergic receptor P2×3, ligand-gated ion channel; calcium channel, alpha 1 beta; sodium channel, beta 1 subunit; neuronal acetylcholine receptor protein alpha 7 subunit precursor (CHRNA7; ACRA7); neuronal nicotinic acetylcholine receptor alpha 2 subunit; proton gated cation channel drasic; sensory neuron specific; channel-inducing factor precursor (CHIF); corticosteroid-induced protein; MYELIN BASIC PROTEIN S (MBP S); organic cation transporter 2 (OCT2); ASIC1 proton gated cation channel; glycine receptor alpha 3 subunit precursor (GLRA3); voltage-gated K+ channel protein; RK5; potassium channel protein; voltage-activated calcium channel alpha-I subunit (RBE-II); nickel-sensitive T-type calcium channel alpha-1 subunit; inward rectifier potassium channel subfamily J member 2 (KCNJ2); RBL-IRK1; eek proto-oncogene, protein tyrosine kinase, eph/elk-related; prostaglandin D2 receptor; activin receptor type I precursor (ACVR1; ACTR1); serine/threonine-protein kinase receptor R1 (SKR1); TGF-B superfamily receptor type I (TSR-I); ACVRLK2; calcitonin receptor precursor (CT-R); C1A/C1B; prostaglandin E2 receptor EP2 subtype (PGE receptor EP2 subtype; PTGER2); prostanoid EP2 receptor; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; gastrin-releasing peptide precursor (GRP); neuromedin C; serotonin receptor; 5-hydroxytryptamine 6 receptor (5-HT-6); ST-B17; possesses high affinity for tricyclic psychotropic drugs; platelet activating factor receptor; alpha 2B adrenergic receptor (ADRA2B); alpha 2B adrenoceptor; VASOACTIVE INTESTINAL POLYPEPTIDE RECEPTOR 2 PRECURSOR (VIP-R-2) (PITUITARY ADENYLATE CYCLASE ACTIVATING POLYPEPTIDE TYPE III RECEPTOR) (PACAP TYPE III RECEPTOR) (PACAP-R-3).; transforming growth factor beta 3 (TGF-beta3); antiproliferative growth factor; vasopressin V1b receptor; prostaglandin E2 receptor EP4 subtype; alpha 2C adrenergic receptor (ADRA2C); alpha 2C adrenoceptor; vasopressin/arginine receptor, V1a; prostaglandin F2 alpha receptor; growth hormone secretagogue receptor 1 (GHSR); cholecystokinin receptor; NMDAR2A N-METHYL-D-ASPARTATE RECEPTOR SUBUNIT; P2U PURINOCEPTOR 1 (ATP RECEPTOR) (P2U1) (PURINERGIC RECEPTOR).; estrogen receptor beta (ER-beta); ESR2; NR3A2; kappa-type opioid receptor (KOR-1); lutropin-choriogonadotropic hormone receptor; beta 1 adrenergic receptor (ADRB 1 R); 5-hydroxytryptamine (serotonin) receptor 1 B; 5-HT1B; adrenergic receptor, beta 2; muscarinic acetylcholine receptor M3 (MACHR); B1 bradikinin receptor; mu opioid receptor (MUOR1); mu-type opioid receptor (MOR-1); opioid receptor B; serotonin 5HT2 receptor; somatostatin receptor 2; melatonin receptor; somatostatin receptor; galanin receptor 1; neuromedin B receptor; transmembrane receptor UNC5H1.; pancreatic polypeptide receptor PP1; interleukin-2 (IL-2); somatostatin; luteinizing hormone, alpha; mast cell protease 1 precursor (RMCP-1); secretory protein probasin (M-40); E-selectin precursor; endothelial leukocyte adhesion molecule 1 (ELAM-1); leukocyte-endothelial cell adhesion molecule 2 (LECAM2); CD62E; Protein kinase C-binding protein beta15; RING-domain containing; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; Wilms' tumor protein (WT1); tumor suppressor; CD28, T-cell surface antigen; c-fgr proto-oncogene; CD3, gamma chain; cathepsin E; S-myc proto-oncogene protein; myc-related; G protein-activated inward rectifier potassium channel 4 (GIRK4); inward rectifier potassium channel subfamily J member 5 (KCNJ5); heart KATP channel; KATP-1; cardiac inward rectifier (CIR); KIR3.4; fructose (glucose) transporter; sodium channel protein 6 (SCP6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; ATPase, sodium/potassium, gamma subunit; G protein-activated inward rectifier potassium channel 1 (GIRK1); inward rectifier potassium channel subfamily J member 3 (KCNJ3); KGA; KGB1; KIR3.1; proton gated cation channel drasic; sensory neuron specific; sodium channel 2, brain; ATPase, copper-transporting, Menkes protein; channel-inducing factor precursor (CHIF); corticosteroid-induced protein; synaptotagmin II; carbonic anhydrase 4; calcitonin receptor precursor (CT-R); C1A/C1B; vasopressin V2 receptor; 5-hydroxytryptamine (serotonin) receptor 1B; 5-HT1B; gamma-aminobutyric acid receptor alpha 4 subunit precursor (GABA(A) receptor; GABRA4); vitamin D3 receptor (VDR); 1,25-dihydroxyvitamin D-3 receptor; NR1I1; muscarinic acetylcholine receptor M5 (CHRM5); somatostatin receptor; galanin receptor 1; granulocyte-macrophage colony-stimulating factor (GM-CSF); colony-stimulating factor (CSF); guanylyl cyclase (membrane form); parathyroid hormone receptor PTH2; galanin receptor 2; 5-hydroxytryptamine (serotonin) receptor 2B; guanine nucleotide-binding protein G(I)/G(S)/G(O) gamma-7 subunit (GNG7; GNGT7); adenylyl cyclase 4; protein kinase C-binding protein nel homolog 1; phospholipase C beta 3 (PLC-beta 3); tissue-type plasminogen activator (t-PA); NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; T-cell receptor CD3 zeta subunit; P-selectin precursor; granule membrane protein 140 (GMP-140); PADGEM; CD62P; leukocyte-endothelial cell adhesion molecule 3 (LECAM3); T-cell receptor gamma subunit; kidney band 3 anion exchange protein; SLC4A1; AE1; L-selectin precursor; lymph node homing receptor; leukocyte adhesion molecule-1 (LAM-1); LY-22; lymphocyte surface MEL-14 antigen; leukocyte-endothelial cell adhesion molecule 1 (LECAM1); CD62L; myelin P0 protein precursor; MPZ; MAL; T-lymphocyte maturation-associated protein; myelin protein MVP17; ErbB3 EGF receptor-related proto-oncogene; HER3; CD 30L receptor; lymphocyte activation antigen CD30; Ki-1 antigen; CD30 precursor; zinc transporter (ZnT-1); CCHB3; calcium channel (voltage-gated; DIHYDROPYRIDINE-SENSITIVE L-TYPE, CALCIUM CHANNEL BETA-3 SUBUNIT.; water channel aquaporin 3 (AQP3); 3-methylcholanthrene-inducible cytochrome P450 (P450MC); cytochrome P450 IA1 (CYPIA1); sodium/potassium-transporting ATPase beta 1 subunit (ATP1B1); glucose transporter 3; ATP-sensitive inward rectifier potassium subfamily J member 8 (KCNJ8); UKATP-1; ATP-sensitive inwardly rectifying K+ channel KIR6.1; RIM; Rab3 effector in synaptic-vesicle fusion; neuronal acetylcholine receptor protein alpha-3 chain precursor; purinergic receptor P2X5, ligand-gated ion channel; sodium channel I; renal organic anion transporter (ROAT1)+multispecific organic anion transporter (OAT1); neuronal acetylcholine receptor protein alpha 6 subunit precursor (CHRNA6; ACRA6); sodium channel, beta 1 subunit; sodium-hydrogen exchange protein-isoform 2 (NHE-2); PMCA; ATP2B2; calcium-transporting ATPase plasma membrane (brain isoform 2; EC 3.6.1.38); calcium pump; fibrinogen beta subunit (FGB); sulfonylurea receptor (SUR); glycine receptor alpha 3 subunit precursor (GLRA3); multidrug resistance protein 2 (MDR2); P-glycoprotein (PGY2); potassium channel, voltage gated, KV3.4; RAW3; KCNC4; sodium/chloride cotransporter, thiazide sensitive; synaptosomal associated protein 25; SNAP-25; SNAP; SNAP25; SUP; calcitonin receptor precursor (CT-R); C1A/C1B; gamma-aminobutyric acid (GABA-A) receptor, beta 1 subunit; NEUREXIN I-BETA PRECURSOR, Non-processed neurexin I-beta Synaptic cell surface proteins+NEUREXIN I-ALPHA PRECURSOR, Non-processed neurexin I-alpha Synaptic cell surface proteins; alpha 2B adrenergic receptor (ADRA2B); alpha 2B adrenoceptor; neuropeptide Y receptor type 1; prostaglandin E2 receptor EP4 subtype; alpha 2C adrenergic receptor (ADRA2C); alpha 2C adrenoceptor; c-ErbA oncogene; thyroid hormone receptor alpha-1 (THRA1); gamma-aminobutyric acid receptor alpha 2 subunit precursor (GABA(A) receptor; GABRA2); P2Y PURINOCEPTOR 6 (P2Y6); glutamate receptor 1 precursor (GluR-1); GluR-A; GluR-K1; gamma-aminobutyric acid receptor alpha 3 subunit precursor (GABA(A) receptor; GABRA3); NMDAR2A N-METHYL-D-ASPARTATE RECEPTOR SUBUNIT; P2U PURINOCEPTOR 1 (ATP RECEPTOR) (P2U1) (PURINERGIC RECEPTOR).; 5-hydroxytryptamine (serotonin) receptor 1B; 5-HT1B; glycine receptor, alpha 2A subunit, inhibitory; parathyroid hormone receptor PTH2; 5-hydroxytryptamine 5A receptor (5HT5A; HTR5A); serotonin receptor; REC17; acetylcholine receptor alpha; brain natriuretic peptide (BNP); 5-kDa cardiac natriuretic peptide; ISO-ANP; luteinizing hormone, alpha; cocaine/amphetamine-induced rat transcript, CART; protein kinase C-binding protein nel homolog 1; 14-3-3 protein eta; PKC inhibitor protein-1; KCIP-1; plectin; NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP).; syndecan 3; ras-GTPase-activating protein (GAP); ras p21 protein activator; p120GAP; interleukin-6 receptor beta chain; membrane glycoprotein gp130; prostatic secretory protein probasin (M-40); A-raf proto-oncogene; prothymosin-alpha (PTMA); cadherin 6 precursor; kidney-cadherin (K-cadherin); neurofibromin; neurofibromatosis protein type I (NF1); GTPase stimulatory protein; c-H-ras proto-oncogene; transforming G-protein p21; HSP84; HSP90-beta; heat shock 90 kD protein; Neural adhesion molecule F3, RAT NEURAL ADHESION MOLECULE F3, COMPLETE CDS.; BIG-1 PROTEIN PRECURSOR; neural cell adhesion protein; neurite outgrowth-promotor; potassium channel protein; KSHIIIA3; ATP-sensitive inward rectifier potassium channel subfamily J member 1 (KCNJ1); KAB-1; KIR1.1; ROMK1; Band 3 (B3RP3), 3 Cl-HCO3-anion exchanger; voltage-gated potassium channel protein KV11.1; RBK1; RCK1; KCNA1; potassium channel, inward rectifier 9; taurine transporter; neuronal acetylcholine receptor protein alpha-3 chain precursor; sodium channel I; potassium channel protein CDRK; neuronal acetylcholine receptor protein alpha 6 subunit precursor (CHRNA6; ACRA6); calcium channel, alpha 1 beta; sodium channel, beta 1 subunit; PMCA; ATP2B2; calcium-transporting ATPase plasma membrane (brain isoform 2; EC 3.6.1.38); calcium pump; 17-kDa ubiquitin-conjugating enzyme E2 (UBE2B); ubiquitin-protein ligase; ubiquitin carrier protein; HR6B; synaptosomal associated protein 25; SNAP-25; SNAP; SNAP25; SUP; 67-kDa glutamic acid decarboxylase (GAD67); GAD1; eek proto-oncogene, protein tyrosine kinase, eph/elk-related; D(1A) DOPAMINE RECEPTOR; growth hormone receptor precursor (GH receptor; GHR); serum-binding protein; NMDAR2A N-METHYL-D-ASPARTATE RECEPTOR SUBUNIT; 5-hydroxytryptamine (serotonin) receptor 1B; 5-HT1B; thyroid hormone beta receptor; c-erbA-beta; gamma-aminobutyric acid (GABA-A) receptor, beta 3 subunit; glutamate receptor 2 precursor (GLUR-2; GLUR-B; GLUR-K2); glutamate receptor 4 precursor (GLUR-4; GLUR-D); cannabinoid receptor 1, neuronal; neuromedin K receptor (NKR); neurokinin B receptor; NK-3 receptor (NK3R); GABA-A receptor gamma-2 subunit precursor; galanin receptor 2; insulin-like growth factor binding protein 1 precursor (IGFBP-1; IBP-1); presomatotropin; protein kinase C beta-I type (PKC-beta I)+protein kinase C beta-II type (PKC-beta II); guanine nucleotide-binding protein G(O) alpha subunit (GNAO; GNA0); guanine nucleotide-binding protein G(I) alpha 1 subunit (GNAI1); adenylate cyclase-inhibiting G alpha protein; serine/threonine kinase PCTAIRE2 (PCTK2); protein kinase C-binding protein nel homolog 1; PKI-alpha; cAMP-dependent protein kinase inhibitor (muscle/brain form); 14-3-3 protein eta; PKC inhibitor protein-1; KCIP-1; and NVP; neural visinin-like Ca2+-binding protein, VISININ-LIKE PROTEIN 1 (VILIP-1) (NEURAL VISININ-LIKE PROTEIN 1) (NVL-1) (NVP-1) (21 KD CABP), with a test compound;
determining activity of said one or more NM proteins in said cells; and
identifying a test compound as a candidate drug for treating neuronal cell death if it increases the activity of one more NM proteins in said cells.

52. The method of claim 51 wherein the cells are retinal cells.

53. The method of claim 51 wherein the cells are recombinant host cells which are transfected with an expression construct which encodes said one or more NMs.
